(19) United States
(12) Patent Application Publication
(10) Pub. No.: US 2006/0089316 A1
(43) Pub. Date: Apr. 27, 2006

(54) Method for reducing a susceptibility to tumor formation induced by 3-deoxyglucosone and precursors thereof

(76) Inventors: Truman R. Brown, Cornwall-on-Hudson, NY (US); Francis Kappler, Philadelphia, PA (US); Steven H. Seeholzer, Huntingdon Valley, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/972,971

(22) Filed: Oct. 25, 2004

(51) Int. Cl. A61K 31/70; A61K 31/60; A61K 31/5377; A61K 31/53; A61K 31/44; A61K 31/415

(52) U.S. Cl. 514/23; 514/242; 514/256; 514/275; 514/639; 514/352; 514/235.5; 514/383; 514/394; 514/396; 514/561; 514/620; 514/567; 514/159; 514/406; 514/473; 514/425

(57) ABSTRACT

Disclosed are methods of using various compounds, which are known to bind to 3-deoxyglucosone (3DG) or precursors thereof, in order to reduce a susceptibility to tumor formation and/or to prevent or delay onset of tumor formation induced by 3DG and its precursors. Also disclosed is the reduction of 3DG levels in high fructose corn syrop so that the high fructose corn syrup is less likely to induce tumor formation.

### FIELD OF THE INVENTION

The present invention relates to a new therapeutic use of various known classes of compounds that have the ability to complex or otherwise bind to reactive carbonyl moieties of compounds formed from the cleavage of early stage glycosylation products. These compounds have been reported to be effective for treatment of various disease states, including retinopathy, cataracts, diabetic kidney disease, glomeruloscierosis, peripheral vascular disease, arteriosclerosis obliterans, peripheral neuropathy, stroke, hypertension, atherosclerosis, osteoarthritis, periarticular rigidity, loss of elasticity and wrinkling of skin, stiffening of joints, and glomerulonephritis. More specifically, it has now been discovered that the compounds described hereinbelow, because of their effectiveness in binding such reactive carbonyl-containing compounds, are also useful in reducing a susceptibility to tumor formation, as well as in preventing or delaying the onset of tumor formation.

### BACKGROUND OF THE PRIOR ART

The role of 3-DG and related metabolites in contributing to human disease has been previously investigated as will be appreciated from a review of the patents listed below. The listed patents are generally directed to compositions and methods for inhibiting non-enzymatic protein aging. The compositions described in these patents comprise various active agents which have in common the capability of inhibiting the formation of advanced glycosylation end products (AGEs) of target proteins by reacting with carbonyl compounds, such as glycoaldehyde, glyceraldehyde or 3-deoxyglucosone, formed from the cleavage of Amadori or other “early glycosylation product(s)”, as defined in those patents. The compounds described in the listed patents presumably act by complexing 3DG, thereby preventing its reaction with proteins, one of the irreversible steps of AGE-protein formation. The disclosures provided in the listed patents do not suggest the possibility of inhibiting the formation of 3DG. They focus exclusively on complexing this toxic molecule. Certain of the compounds described in the listed patents, for example, U.S. Pat. No.5,262,152, are α-effect amines, which are known to react with dicarbonyl compounds, such as 3DG. See, W. P. Jencks, 3^{rd} ed., McGraw Hill, New York.

| U.S. Pat. No. | Active Agent |
|---|---|
| 5,698,563 to Wagle et al. | bis(guanylhydrazones) |
| 5,661,139 to Lankin et al. | bis-2-(aryl)hydrazones |
| 5,565,261 to Cerami et al. | thiazolium compounds |
| 5,612,332 to Wagle et al. | di- or tri-aminoguanidines |
| 5,534,540 to Ulrich and Wagle | substituted or unsubstituted tetramic |
| | and tetronic acids |
| 5,476,849 to Ulrich et al | amino-benzoic acids and derivatives |
| 5,128,360 to Cerami et al | aminoguanidine |
| 5,468,777 to France et al | cysteine and cysteine derivatives |
| 5,358,960 to Ulrich et al | aminosubstituted imidazoles |
| 5,334,617 to Ulrich et al | amino acids |
| 5,318,982 to Ulrich et al | 1,2,4-triazoles |
| 5,272,165 to Ulrich et al | 2-alkylidene-aminoguanidines |
| 5,262,152 to Ulrich et al | amidrazones and derivatives |
| 5,258,381 to Ulrich et al | 2-substituted-2-imidazolines |
| 5,243,071 to Ulrich et al | 2-alkylidene-aminoguanidies |
| 5,221,683 to Ulrich et al | diaminopyridine compounds |
| 5,130,324 to Ulrich et al | 2-alkylidene-aminoguanidines |
| 5,114,943 by Ulrich et al | amino-substituted pyrimidines |

Another approach to reducing the deleterious health effects of glycated protein, as manifested in diabetic complications, involves the administration of antibodies or other immunoreactive substances which specifically bind to glycated albumin or its cellular receptor, or to glycated low-density lipoprotein (LDL). See, for example, U.S. Pat. Nos. 5,223,392, 5,494,791 and 5,518,720 to M. Colen. One such antibody is characterized as specifically binding to an epitope present on glycated albumin but not present on non-glycated albumin or other human protein. A representative antibody of this kind is known as A717, which is produced by cell line ATCC HB 9596. Another antibody useful for this purpose is one which reacts immunospecifically with an epitope comprising N-deoxyfructosyllysine, which is present in glycated LDL, but not in unglycated LDL or in other glycated or unglycated plasma proteins, the epitope being present in apolipoprotein B of glycated LDL.

Methods for monitoring metabolic control in diabetic patients by measurement of glycosylation end-products are known. The concentration of glycosylated hemoglobin is known to reflect mean blood glucose concentration during the preceding several weeks. U.S. Pat. No. 4,371,374, issued to A. Cerami et al., describes a method for monitoring glucose levels by quantitation of the degradation products of glycosylated proteins, more specifically non-enzymatically glycosylated amino acids and peptides, in urine. This method purports to utilize the affinity of alkaline boronic acids for forming specific complexes with the coplanar cis-diol groups found in glycosylation end-products to separate and quantitate such end-products.

U.S. Pat. No. 4,761,368 issued to A. Cerami describes the isolation 2 5 and purification of a chromophore present in browned polypeptides, e.g., bovine serum albumin and poly-L-lysine. The chromophore, 2-(2-furoyl)4(5)-2(furoyl)-1H-imidazole (FFI) is a conjugated heterocycle derived from the condensation of two molecules of glucose with two lysine-derived amino groups. This patent further describes the use of FFI in a method for measuring “aging” (the degree of advanced glycosylation) in a protein sample wherein the sample “age” is determined by measuring the amount of the above-described chromophore in the sample and then comparing this measurement to a standard (a protein sample having an amount of FFI which has been correlated to the “age” of the sample).

The disclosures of all of the above-mentioned patents are incorporated by reference in the present specification, as though set forth herein in full.

As can be appreciated from the foregoing summary of the prior art, although a number of therapeutic agents have been proposed for treating various disease states in which there is evidence that one or more early glycosylation products having an etiologic effect, it is not believed that such agents have previously been proposed for use in reducing a susceptibility to, or preventing or delaying the onset of tumor formation.

### SUMMARY OF THE INVENTION

The present invention arose, in part, from the discovery of a metabolic pathway that involves the enzyme-mediated conversion of fructose lysine (FL) to fructose-lysine-3-phosphate (FL3P) and produces relatively high concentrations of 3-deoxyglucosone (3DG) in organs affected by diabetes. See International Patent Application WO 98/33492, published Aug. 6, 1998, the entire disclosure of which is incorporated by reference in the present specification as though set forth herein in full. Further research into the biochemical function of this newly discovered pathway tends to indicate that it also has a role in the etiology of tumor formation. It is now suspected that this pathway contributes to the development of tumor formation, particularly renal cell carcinoma.

This discovery has found practical application in the present invention which, in one aspect, provides a method of reducing a suceptibility to tumor formation induced by the presence of 3DG in a subject by administering to the subject at least one of the therapeutic agents identified below in the detailed description of the invention. Also in accordance with the present invention a method is provided for preventing or delaying the onset of tumor formation caused by 3DG. The method comprises administering a therapeutic amount of an agent that counteracts the deleterious effects of 3DG that contribute to tumor formation.

According to another aspect, this invention provides a method of making high fructose corn syrup less likely to induce tumor formation, by treating 3DG-containing high fructose corn syrup to reduce the 3DG levels thereof to below the normal level of 3DG present in human plasma, which is in the range from about 50 to about 100 nM. The resultant high fructose corn syrup has a 3DG content of less than 0.1 μM.

### BRIEF DESCRIPTION OF DRAWINGS

FIG. 1 illustrates the reactions involved in the lysine recovery is pathway.

### DETAILED DESCRIPTION OF THE INVENTION

The following definitions are provided to facilitate understanding of the present invention, as described in further detail hereinbelow:

1. Glycated-Lysine Residues—The expression “glycated lysine residues”, as used herein, refers to the modified lysine residue of a stable adduct produced by the reaction of a reducing sugar and a lysine-containing protein.

The majority of protein lysine residues are located on the surface of proteins as expected for a positively charged amino acid. Thus, lysine residues on proteins which come in contact with serum, or other biological fluids, can freely react with sugar molecules in solution. This reaction occurs in multiple stages. The initial stage involves the formation of a Schiff base between the lysine free amino group and the sugar keto-group. This initial product then undergoes the Amadori rearrangement, to produce a stable ketoamine compound.

This series of reactions can occur with various sugars. When the sugar involved is glucose, the initial Schiff base product will involve imine formation between the aldehyde moiety on C-1 of the glucose and the lysine E-amino group. The Amadori rearrangement will result in formation of lysine coupled to the C-1 carbon of fructose, 1-deoxy-1-(ε-aminolysine)-fructose, herein referred to as fructose-lysine or FL.

Similar reactions will occur with other aldose sugars, for example galactose and ribose (Dills, Am. J. Clin. Nutr., 58: S779 (1993)). For the purpose of the present invention, the early products of the reaction of any reducing sugar and the ε-amino residue of protein lysine are included within the meaning of glycated-lysine residue, regardless of the exact structure of the modifying sugar molecule.

Also, the terms glycated-lysine residue, glycated protein and glycosylated protein or lysine residue are used interchangeably herein, which is consistent with current usage in scientific journals where such expressions are often used interchangeably.

2. Fructose-lysine—The term “fructose-lysine” (FL) is used herein to signify any glycated-lysine, whether incorporated in a protein/peptide or released from a protein/peptide by proteolytic digestion. This term is specifically not limited to the chemical structure commonly referred to as fructose-lysine, which is reported to form from the reaction of protein lysine residues and glucose. As noted above, lysine amino groups can react with a wide variety of sugars. Indeed, one report indicates that glucose is the least reactive sugar out of a group of sixteen (16) different sugars tested (Bunn et al., Science, 213: 222 (1981)). Thus, tagatose-lysine formed from galactose and lysine, analogously to glucose, is included wherever the term fructose-lysine is mentioned in this description, as is the condensation product of all other sugars, whether naturally-occurring or not. It will be understood from the description herein that the reaction between protein-lysine residues and sugars involves multiple reaction steps. The final steps in this reaction sequence involve the crosslinking of proteins and the production of multimeric species, known as AGE-proteins, some of which are fluorescent. Proteolytic-digestion of such modified proteins does not yield lysine covalently linked to a sugar molecule. Thus, these species are not included within the meaning of “fructose-lysine”, as that term is used herein.

3. Fructose-lysine-3-phosphate—This compound is formed by the enzymatic transfer of a high energy phosphate group from ATP to FL. The term fructose-lysine-3-phosphate (FL3P), as used herein, is meant to include all phosphorylated fructose-lysine moieties that can be enzymatically formed whether free or protein-bound.

4. Fructose-lysine-3-phosphate kinase—This term refers to one or more proteins which can enzymatically convert FL to FL3P, as defined above, when additionally supplied with a source of high energy phosphate.

5. 3-Deoxyglucosone-3-Deoxyglucosone (3DG) is the 1,2-dicarbonyl-3-deoxysugar (also known as 3-deoxyhexulosone) which is formed upon breakdown of FL3P to yield free lysine and inorganic phosphate. For purposes of the present description, the term 3-deoxyglucosone is intended to include all possible dicarbonyl sugars which are formed upon breakdown of FL3P, having the broad definition of FL3P stated above.

6. FL3P Lysine Recovery Pathway—A lysine recovery pathway exists in human kidney, and possibly other tissues, which regenerates unmodified lysine as a free amino acid or incorporated in a polypeptide chain. As explained below, the metabolites produced by this pathway induce a susceptibility to carcinoma.

7. “Early glycosylation product(s)”—This term is intended to encompass any and all early glycosylation products with which the compounds described below are capable of reacting, including, without limitation, early glycosylation products with carbonyl moieties that are involved in the formation of advanced glycosylation end products and that are bound by interaction with the compounds described below. Such products may comprise reactive carbonyl moieties of Amadori products or their further condensation, dehydration and/or rearrangement products, which may condense to form advanced glycosylation end products. Also, reactive carbonyl compounds, containing one or more carbonyl moieties (such as glycoladehyde, glyceraldehyde or 3DG) may form from the cleavage of Amadori or other glycosylation end products, and by subsequent reactions with an amine or Amadori products, may form carbonyl-containing advanced glycosylation products such as alkylformyl-glycosylpyrroles.

8. “Susceptible test animal”—As used herein this expression refers a strain of laboratory animals which, due to the presence of certain genetic mutations have a higher propensity towards malignant transformation and tumor formation. The Eker rat, which has a mutation in the tuberous sclerous gene (Tsc-2), is one example of such a susceptible test animal. One of ordinary skill in the art is no doubt aware of a variety of other laboratory rat or mouse strains with increased propensity for tumor formation. The phrase “similar susceptible test animal” refers to animals of a comparable genetic background which are used as control, untreated animals.

The present invention evolved from the discovery of a previously unknown metabolic pathway, referred to herein as the FL3P lysine recovery pathway, which produces 3DG in an enzyme-catalyzed reaction. This enzymatic pathway is capable of enzymatic inhibition, thereby reducing the production of toxic 3DG.

Previously, it had been generally accepted that the origin of 3DG in vivo was from the decomposition of proteins containing glycated lysine residues. It had also been commonly believed that these glycated-lysines could not be used as an amino acid source. Additional extensive laboratory investigation has now shown that this previous belief was incorrect. See, WO 98/33492, supra.

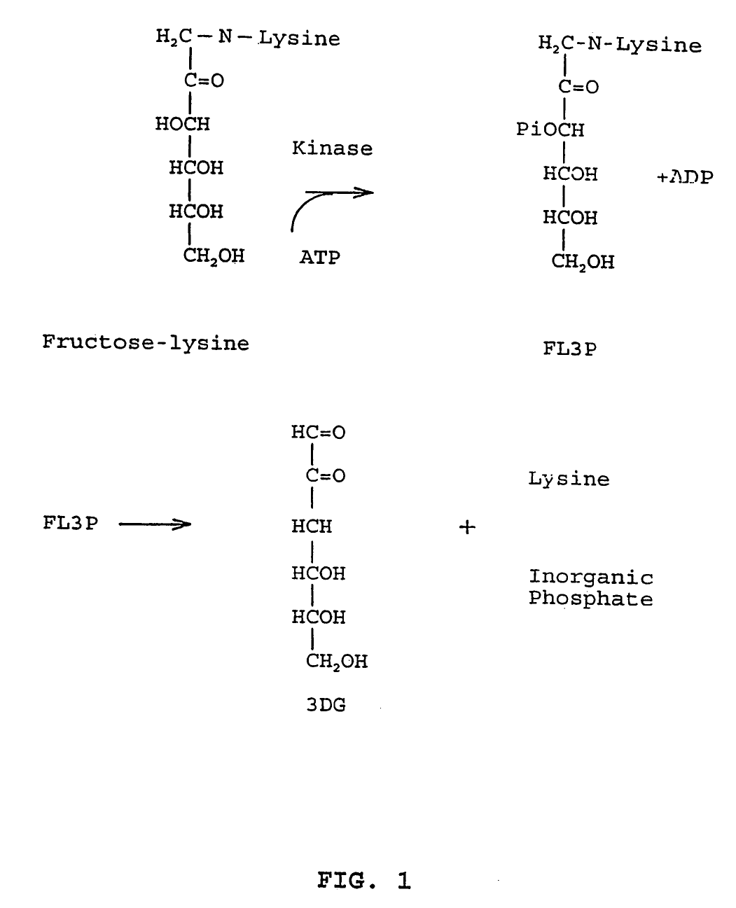

The reaction steps involved in this newly discovered metabolic pathway are presented in FIG. 1. In the first step, fructose-lysine and ATP react to form FL3P and ADP in a reaction catalyzed by FL3P kinase. Phosphorylation occurs on the 3-position of the fructose moiety, leading to destabilization of the fructoselysine molecule. The resulting FL3P then decomposes to form 3DG, inorganic phosphate, and unmodified, free, reusable lysine, which is available for utilization in protein synthesis. Aldehyde reductase detoxifies 3DG by reduction to 3-deoxyfructose (3DF), which is excreted in urine.

FL3P is not stable in aqueous solution. It rapidly degrades to form 3DG, lysine and inorganic phosphate. This reaction also occurs in vivo. It is not currently know if the degradation of FL3P that occurs in vivo is a spontaneous or enzyme catalyzed reaction. It is strongly suspected, however, that enzymatic catalysis is involved, as the production of 3DG from fructose-lysine occurs very rapidly in intact kidney.

Although FIG. 1 illustrates the FL3P lysine recovery pathway using the most prevalent glycated-lysine, i.e., fructose-lysine, it will be readily apparent to those skilled in the art that a wide variety of similar molecules can flux through this pathway. Indeed, the substrate selectivity of the FL3P lysine recovery pathway is quite broad, thus warranting the broad definition of the terms given above.

Additional experiments have shown that the lysine recovery pathway is found in a wide variety of warm-blood vertebrate species, including sheep, pig, dog, rabbit, cow, mice and chicken. This pathway is also present in humans. See, WO 98/33492, supra. The ubiquitous presence of the FL:3P lysine recovery pathway can be understood, given that lysine is an essential amino acid which is present in relatively low concentrations in most foods. In addition, an appreciable percentage of the lysine residues in food will exist in the glycated form and the proportion of this modified lysine will increase when the food is cooked. Since these glycated lysine residues can not be utilized for protein synthesis, a recovery pathway for lysine is of great utility and affords a selective advantage to organisms which possess it.

A method of reducing susceptibility to tumor formation in a patient associated with the intake of glycated protein is the subject of related U.S. application Ser. No. 09/182,114, filed Oct. 28, 1998, the entire disclosure of which is incorporated by reference in the present specification as though set forth herein in full. That method comprises the administration of a pharmaceutical composition which contains an active compound having inhibitory activity for the enzymatic conversion of fructose-lysine to FL3P. Another aspect of that invention concerns a method of preventing or delaying the onset of tumor formation caused by the formation of AGE-proteins, which comprises administering a therapeutic amount of an agent that inhibits production of 3DG.

In accordance with the present invention, other approaches for reducing a susceptibility to tumor formation, and for preventing or delaying the onset of tumor formation have been devised. One such approach involves administering an agent which is effective to bind 3DG in the system of a subject undergoing treatment in accordance with this invention. The agents which are suitable for this purpose are described hereinbelow. These agents are small molecules that are effective in binding 3DG, irrespective of whether the 3DG is biochemically formed by the subject, e.g., via the FL3P lysine recovery pathway, or is present in food or beverage ingested by the subject which raises endogenous levels of 3DG.

Another approach entails binding of substrates that function as precursors of 3DG, for example, FL or analogous compounds that are metabolized in vivo to form 3DG. This method is preferably carried out using an immunoreactive agent that specifically binds 3DG precursors in vivo. Representative examples of such immunoreactive agents include the monoclonal antibody produced by cell line ATCC HB 9596, which specifically binds an epitope present on glycated albumin. Substrates which may be bound in this manner so as to interfere with the production of 3DG include any of the phosphorylated fructose-lysine moieties, whether free or protein-bound, that can be enzymatically produced in the system of a subject undergoing treatment. Antibodies may be generated, using well-known procedures, to a variety of substrates that may vary by size, including, without limitation, amino acid-fragments, peptide-lysine fragments, polypeptide fragments, and the like.

The agents involved in the lysine recovery pathway have been identified in other tissues besides kidney, specifically red blood cells, lens, and peripheral nerve tissues. These agents are also found in pancreas tissue.

Compounds which may be used in the practice of this invention include one or more (i.e., combinations) of the following:

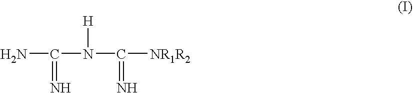

wherein R₁ and R₂ are independently hydrogen, lower alkyl, lower alkoxy or an aryl group; or together with the nitrogen atom form a heterocyclic ring containing from 1 to 2 heteroatoms and 2 to 6 carbon atoms, the second of said heteroatoms being selected from the group consisting of nitrogen, oxygen and sulfur; and their biocompatible and pharmaceutically acceptable acid addition salts.

The lower alkyl groups in the compounds of Formula (I) contain 1-6 carbon atoms and include methyl, ethyl, propyl, butyl, pentyl, hexyl, and the corresponding branched chain isomers thereof The lower alkoxy groups have 1-6 carbon atoms and include methoxy, ethoxy, propoxy, butoxy, penthyloxy, hexyloxy and branched chain isomers thereof. The aryl groups include both substituted and unsubstituted phenyl and pyridyl groups. Typical aryl group substituents are those such as lower alkyl groups, fluoro, chloro, bromo and iodo atoms.

Of the compounds encompassed by Formula I, certain combinations of substituents are preferred. For instance, when R₁ is a hydrogen atom, then R₂ is preferably hydrogen or an aryl group.

When R₁ and R₂ are both alkyl groups, then the compounds having identical R₁ and R₂ alkyl groups are preferable.

When R₁ and R₂ together with the nitrogen atom form a heterocyclic ring containing from 1 to 2 heteroatoms, said heteroatoms being selected from the group consisting of nitrogen, oxygen and sulfur, the preferred heterocyclic rings will be morpholino, piperazinyl, piperidinyl and thiomorpholino, with the morpholino being most preferred.

Representative of the compounds of formula (I) are:
N,N-dimethylimidodicarbonimidic diamide; imidodicarbonimidic diamide;
N-phenylimidodicarbonimidic diamide;
N-(aminoiminomethyl)4-morpholinecarboximidamide;
N-(aminoiminomethyl)4-thiomorpholinecarboximidamide;
N-(aminoiminomethyl)4-methyl-1-piperazinecarboximidamide;
N-(aminoiminomethyl)-1-piperidinecarboximidamide;
N-(aminoiminomethyl)-1-pyrrolidinecarboximidamide;
N-(aminoiminomethyl)-1-hexahydroazepinecarboximidamide;
N-4-pyridylimidodicarbonimidic diamide;
N,N-di-n-hexylimidodicarbonimidic diamide;
N,N-di-n-pentylimidodicarbonimidic diamide;
N,N-d-n-butylimidodicarbonimidic diamide;
N,N-dipropylimidodicarbonimidic diamide;
N,N-diethylimidodicarbonimidic diamide;
and the pharmaceutically acceptable acid addition salts thereof;

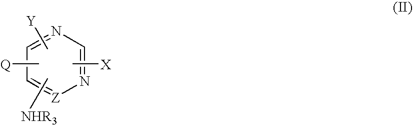

wherein Z is N or CH—;

X, Y and Q are each independently a hydrogen, amino, heterocyclo, amino lower alkyl, lower alkyl or hydroxy group; and

R₃ is hydrogen or an amino group; and their corresponding 3-oxides; an their biocompatible and pharmaceutically acceptable salts.

The compounds of Formula II wherein the X, Y or Q substituent is on a nitrogen of the ring exist as tautomers, i.e., 2-hydroxypyrimidine can exist also as 2(1H)-pyrimidine. Both forms may be used in practicing this invention.

The lower alkyl groups of the compounds of formula II contain 1-6 carbon atoms and include methyl, ethyl, propyl, butyl, pentyl, hexyl, and the corresponding branched chain isomers thereof. The heterocycylic groups of the compounds of formula II contain from 3-6 carbon atoms and are exemplified by groups such as pyrrolidinyl, 2-methylpyrrolidinyl, piperidinol, 2-methylpiperidino morpholino, and hexamethyleneamino.

The “floating” X, Y, Q and NHR₃ bonds in Formula II indicate that these variants can be attached to the ring structure at any available carbon juncture. The hydroxy variant of X, Y and Q can also be present on a nitrogen atom.

Of the compounds encompassed by Formula II, certain combinations of substituents are preferred. For instance, compounds having R₃ as hydrogen, as a CH group, and at least one of X, Y or Q as another amino group, are preferred. The group of compounds where R₃ is hydrogen, Z is a CH group and one of X or Y is an amino lower alkyl group are also preferred. Another preferred group of compounds is those where R is hydrogen and Z is N (nitrogen). Certain substitution patterns are preferred, i.e., the 6-position (IUPAC numbering, Z.dbd.CH) is preferably substituted, and most preferably by an amino or a nitro containing group. Also preferred are compounds where two or more of X, Y and Q are other than hydrogen.

Representative of the compounds of formula II are:
2-hydrazino4-hydroxy-6-methylpyrimidine;
4,5-diaminopyrimidine;
4-amino-5-aminomethyl-2-methylpyrimidine;
6-(piperidino)-2,4-diaminopyrimidine 3-oxide;
3-amino-6-methyl-1,2,4-triazin-5(2H)-one;
4,6-diaminopyrimidine;
4,5,6-triaminopyrimidine;
4,5-diamino-6-hydroxypyrimidine:
2,4,5-triamino-6-hydroxypyrimidine;
5,6-diamino-2,4-dihyroxypyrimidine;
2,4,6-triaminopyrimidine;
4,5-diamino-2-methylpyrimidine;
4,5-diamino-2,6-dimethylpyrimidine;
4,5-diamino-2-hydroxypyrimidine;
4,5-diamino-2-hydroxy-6-methylpyrimidine;
2-hydrazinopyrimidine;
4,6-dimethyl-2-hydrazinopyrimidine;
3-hydrazino-1,2,4-triazine;
3-hydrazino-5-hydroxy-1,2,4-triazine;
5-hydrazino-3-hydroxy-1,2,4-triazine; and
5,6-diamino-3-hydroxy-1,2,4-triazine;

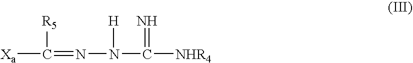

wherein R₄ is hydrogen or acyl;

R₅ is hydrogen or lower alkyl;

Xₐ is a substituent selected from the group consisting of lower alkyl, carboxy, carboxymethyl, or a phenyl or pyridyl group, optionally substituted by halogen, lower alkyl, hydroxy lower alkyl, hydroxy, or acetylamino with the proviso that when X is a phenyl or pyridyl group, optionally substituted, then R₂ is hydrogen; and their biocompatible and pharmaceutically acceptable acid addition salts.

The lower alkyl groups in the compounds of Formula III contain 1-6 carbon atoms and include methyl, ethyl, propyl, butyl, pentyl, hexyl, and the corresponding branched chain isomers thereof. The halo variants can be fluoro, chloro, bromo or iodo substituents.

Equivalent to the compounds of Formula III for the purpose of this invention are the biocompatible and pharmaceutically acceptable salts thereof. Such salts can be derived from a variety of organic and inorganic acids including but not limited to methanesulfonic, hydrochloric, toluenesulfonic, sulfuric, maleic, acetic and phosphoric acids.

Of the compounds encompassed by Formula III, certain substituents are preferred. For instance, R₄ is preferably a methyl group and Xₐ is preferably a phenyl or substituted phenyl group.

Representative of the compounds of Formula III are:
N-acetyl-2-(phenylmethylene)hydrazinecarboximidamide;
2-(phenylmethylene)hydrazinecarboximidamide;
2-(2,6-dichlorophenylmethylene)hydrazinecarboximidamide pyridoxal guanylhydrazone;
pyridoxal phosphate guanylhydrazone;
2-( 1-methylethylidene)hydrazinecarboximidamide;
pyruvic acid guanylhydrazone;
4-acetamidobenzaldehyde guanylhydrazone;
4-acetamidobenzaldehyde N-acetylguanylhydrazone;
acetoacetic acid guanylhydrazone;
and the biocompatible and pharmaceutically acceptable salts thereof;

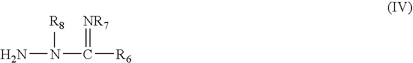

wherein R₆ is hydrogen or a lower alkyl group, or a phenyl group, optionally substituted by 1-3 halo, amino, hydroxy or lower alkyl groups; R₇ is hydrogen, a lower alkyl group, or an amino group and R₈ is hydrogen or a lower alkyl group; their biocompatible and pharmaceutically acceptable acid addition salts.

The lower alkyl groups in the compounds of Formula IV contain 1-6 carbon atoms and include methyl, ethyl, propyl, butyl, pentyl, hexyl, and the corresponding branched chain isomers thereof. The halo variants can be fluoro, chloro, bromo or iodo substituents. Where the phenyl ring is substituted, the point or points of substitution may be ortho meta or para to the point of attachment of the phenyl ring to the straight chain of the molecule.

Equivalent to the compounds of Formula IV for the purpose of this invention are the biocompatible and pharmaceutically acceptable salts thereof. Such salts can be derived from a variety of organic and inorganic acids including but not limited to methanesulfonic, hydrochloric, toluenesulfonic, sulfuric, maleic, acetic and phosphoric acids.

Of the compounds encompassed by Formula IV, certain substituents are preferred. For instance, R₆ is preferably a methyl or ethyl group.

Representative of the compound of Formula IV are:
ethanimidic acid hydrazide;
ethanehydrazonic acid hydrazide;
N-methylethanimidic acid hydrazide;
ethanimidic acid 1-methylhydrazide;
formamidrazone (methaniidic acid hydrazide);
propanimidic acid hydrazide;
benzimidic acid, hydrazide;
benzimidic acid, 1-methylhydrazide;
propanehydrazonic acid nydrazide;
n-butanehydrazonic acid hydrazide;
4-methylbenzamidrazone;
N-methylbenzenecarboximidic acid hydrazide;
benzenecarboximidic acid 1-methylhydrazide;
3-chlorobenzamidrazone;
4-chlorobenzamidrazone;
2-fluorobenzamidrazone;
3-fluorobenzamidrazone;
4-fluorobenzamidrazone;
2-hydroxybenzamidrazone;
3-hydroxybenzamidrazone,
4-hydroxybenzamidrazone:
2-aminobenzamidrazone;
benzenecarbohydrazonic acid hydrazide;
benzenecarbohydrazonic acid 1-methylhydrazide; and the biocompatible and—pharmaceutically acceptable salts thereof; 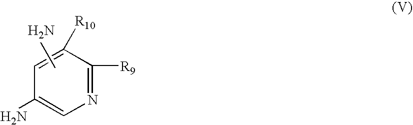
   wherein R₉ and R₁₀ are independently hydrogen, hydroxy, lower alkyl or lower alkoxy; with the proviso that the “floating” amino group is adjacent to the fixed amino group; their biocompatible and pharmaceutically acceptable acid addition salts.

The lower alkyl groups of the compounds of Formula V contain 1-6 carbon atoms and include methyl, ethyl, propyl, butyl, pentyl, hexyl, and the corresponding branched chain isomers thereof. Likewise, the lower alkoxy groups of the compounds of formula V contain 1-6 carbon atoms and include methoxy, ethoxy, propoxy, butoxy pentoxy, hexoxy, and the corresponding branched chain isomers thereof.

Equivalent to the compounds of Formula V for the purpose of this invention are the biocompatible and pharmaceutically acceptable salts thereof. Such salts can be derived from a variety of organic and inorganic acids including but not limited to methanesulfonic, hydrochloric, toluenesulfonic, sulfuric, maleic, acetic and phosphoric acids.

Of the compounds encompassed by Formula V, certain substituents are preferred. For instance, when R₉ is hydrogen then R₁₀ is preferably also hydrogen.

Representative of the compounds of Formula V are:
3,4-diaminopyridine;
2,3-diaminopyridine;
5-methyl-2,3-diaminopyridine;
4-methyl-2,3-diaminopyridine;
6-methyl-2,3-pyridinediamine;
4,6-dimethyl-2,3-pyridinediamine;
6-hydroxy-2,3-diaminopyridine;
6-ethoxy-2,3-diaminopyridine;
6-dimethylamino-2,3-diaminopyridine;
diethyl 2-(2,3-diamino-6-pyridyl)malonate;
6(4-methyl-1-pyperazinyl)-2,3-pyridinediamine;
6-(methylthio)-5-(trifluoromethyl)-2,3-pyridinediamine;
5-(trifluoromethyl)-2,3-pyridinediamine;
6-(2,2,2-trifluorethoxy)-5-(trifluoromethyl)-2,3-pyridinediamine;
6-chloro-5-(trifluoromethyl)-2,3-pyridinediamine;
5-bromo-4,6-dimethyl-2;3-pyridinediamine;
5-methoxy-6-(methylthio)-2,3-pyridinediamine;
5-bromo4-methyl-2,3-pyridinediamine;
5-(trifluoromethyl-2,3-pyridinediamine;
6-bromo-4-methyl-2,3-pyridinediamine;
5-bromo-&methyl-2,3-pyridinediamine;
6-methoxy-3,4-pyridinediamine;
2-methoxy-3,4-pyridinediamine;
5-methyl-3,4-pyridinediamine;
5-methoxy-3,4-pyridinediamine;
5-bromo-3,4-pyridinediamine;
2,3,4-pyridinetriamine;
4,6-dimethyl-2,3,5-pyridinetriamine;
2,3,5-pyridinetriamine;
4-methyl-2,3,6-pyridinetriamine;
4-(methylthio)2,3,6-pyridinetriamine;
4-ethoxy-2,3,6-pyridinetriamine;
2,3,6-pyridinetriamine;
3,4,5-pyridinetriamine;
4-methoxy-2,3-pyridinediamine;
5-methoxy-2,3-pyridinediamine;
6-methoxy-2,3-pyridinediamine;
and the biocompatible and pharmaceutically acceptable salts thereof;

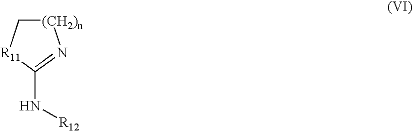

wherein n is 1 or 2;

R₁₁ is an amino group or a hydroxyethyl group; and

R₁₂ is an amino, a hydroxyalkylamino, a lower alkyl group or a group of the formula alk-Yₐ wherein alk is a lower alkylene group and Yₐ is selected from the group consisting of hydroxy, lower alkoxy, lower alkylthio, lower alkylamino and heterocyclic groups containing 4-7 ring members and 1-3 heteroatoms; with the proviso that when R₁₁ is a hydroxyethyl group then R₁₂ is an amino group; their biocompatible and pharmaceutically acceptable acid addition salts.

The lower alkyl, lower alkylene and lower alkoxy groups referred to herein contain 1-6 carbon atoms and include methyl, methylene, methoxy, ethyl, ethylene, ethoxy, propyl, propylene, propoxy, butyl, butylene, butoxy, pentyl, pentylene, pentyloxy, hexyl, hexylene, hexyloxy and the corresponding branched chain isomers thereof. The heterocyclic groups referred to herein include 4-7 membered rings having at least one and up to 3 heteroatoms therein. Representative heterocyclic groups are those such as morpholino, piperidino, piperazino, methylpiperazino, and hexamethylenimino.

Equivalent to the compounds of Formula I for the purpose of this invention are the biocompatible and pharmaceutically acceptable salts thereof. Such salts can be derived from a variety of organic and inorganic acids including but not limited to, methanesulfonic, hydrochloric, toluenesulfonic, sulfuric, maleic, acetic and phosphoric acids.

Of the compounds encompassed by Formula VI, certain combinations of substituents are preferred. For instance, when R₁₁ is a hydroxyethyl group, then R₁₂ is an amino group. When R₁₁ is an amino group, then R₁₂ is preferably a hydroxy lower alkylamino, a lower alkyl group or a group of the formula alk-Y, wherein alk is a lower alkylene group and Y is selected from the group consisting of hydroxy, lower alkoxy, lower alkylthio, lower alkylamino and heterocyclic groups containing 4-7 ring members and 1-3 heteroatoms.

Representative of the compounds of Formula VI are:
1-amino-2-[2-(2-hydroxyethyl)hydrazino]-2-imidazoline;
1-amino-2-(2-hydroxyethylamino)-2-imidazoline;
1-(2-hydroxyethyl)-2-hydrazino-1,4,5,6-tetrahydropyrimidine;
1-(2-hydroxyethyl)2-hydrazino-2-imidazoline;
1-amino-2-([2-(4-morpholino)ethylamino)imidazoline;
1-amino-2-([3-(4-morpholino)propyl]amino)imidazoline;
1-amino-2-([3-(4-methylpiperazin-1-yl)propyl]-amino)imidazoline;
1-amino-2-([3-(dimethylamino)propyl]amino)imidazoline;
1-amino-2-[(3-ethoxypropyl)amino]imidazoline;
1-amino-2-([3-(1-imidazolyl)propyl]amino)imidazoline;
1-amino-2-(2-methoxyethylamino)-2-imidazoline;
1-amino-2-(3-isopropoxypropylamino)-2-imidazoline;
1-amino-2-(3-methylthiopropylamino)-2-imidazoline;
1-amino-2-[3-(1-piperidino)propylamino)imidazoline;
1-amino-2-[2,2-dimethyl-3-(dimethylamino)propylamino]-2-imidazoline;
1-amino-2-(neopentylamino)-2-imidazoline;
and the biocompatible and pharmaceutically acceptable salts thereof;

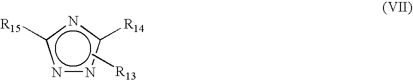

wherein R₁₃ is a hydrogen or an amino group; R₁₄ and R₁₅ are independently an amino group, a hydrazino group, a lower alkyl group, or an aryl group; with the proviso that one of R₁₃, R₁₄ and R₁₅ must be an amino or a hydrazino group; and their biologically or pharmaceutically acceptable acid or alkali addition salts.

The lower alkyl groups referred to above preferably contain 1-6 carbon atoms and include methyl, ethyl, propyl, butyl, pentyl, hexyl, and the corresponding branched-chain isomers thereof.

The aryl groups encompassed by the Formula VII are those containing 6-10 carbon atoms, such as phenyl and lower alkyl substituted-phenyl, e.g. tolyl and xylyl, and phenyl substituted by 1-2 halo, hydroxy or lower alkoxy groups.

The halo atoms in the Formula VII may be fluoro, chloro, bromo or iodo. The lower alkoxy groups contain 1-6, and preferably 1-3, carbon atoms and are illustrated by methoxy, ethoxy, n-propoxy, isopropoxy and the like.

For the purposes of this invention equivalent to the compounds of Formula VII are the biologically and pharmaceutically acceptable acid addition salts thereof. Such acid addition salts may be derived from a variety of organic and inorganic acids such as sulfuric, phosphoric, hydrochloric, hydrobromic, sulfamic, citric, lactic, maleic, succinic, tartaric, cinnamic, acetic, benzoic, gluconic, ascorbic and related acids.

Of the compounds encompassed by Formula VII, certain combinations of substituents are preferred. For instance, when R₁₃ is hydrogen, then R₁₄ is preferably an amino group. When R₁₄ is a hydrazino group, then R is preferably an amino group.

Representative of the compounds of Formula VII are:
3,5-diamino-1,2,4-triazole;
3-methyl-5-amino-1,2,4-triazole;
4-amino-3-hydrazino-5-methyl-1,2,4-triazole;
3,4-diamino-5-methyl-1,2,4-triazole;
4H-1,2,4-triazol-4-amine;
3,5-dimethyl-4H-1,2,4-triazol-4-amine;
3,5-diethyl4H--1,2,4-triazol-4-amine;
3,5-dipropyl4H-1,2,4-triazol4-amine;
3,5-dibutyl-4H-1,2,4-triazol-4-amine;
3,5-dihexyl4H-1,2,4-triazolamine;
3-ethyl4H-1,2,4-triazol-4-amine;
3-methyl-4H-1,2,4-triazol4-amine;
3-ethyl-5-methyl4H-1,2,4-triazol-4-amine;
3-methyl-5-phenyl-4H-1,2,4-triazol-4-amine;
3-pentyl-5-phenyl-4H-1,2,4-triazol-4 amine;
4H-1,2,4-triazole-3,4-diamine;
5-(1-ethylpropyl)4H-1,2,4-triazole-3,4-diamine;
5-isopropyl-4H,1,2,4-triazole-3,4-diamine;
5-cyclohexyl-4H-1,2,4-triazole-3,4diamine;
5-methyl-4H-1,2,4-triazole-3,4-diamine;
5-phenyl4H-1,2,4-triazole-3,4-diamine;
5-propyl4H-1,2,4-triazole-3,4-diamine;
5-cyclohexyl4H-1,2,4-triazole-3,4-diamine;
4-amino-3,5-dihydrazino4H-1,2,4-triazole;
4-amino-3-hydrazino-5-phenyl-4H-1,2,4-triazole;
4-amino-3-hydrazino-5-ethyl-4H-1,2,4-triazole;
3-amino-5-hydrazino-1,2,4-triazole;
3-(2chlorophenyl)-5-hydrazino-1,2,4-triazole;
3-(1-ethylpropyl)5-hydrazino-1,2,4-triazole;
4H-1,2,4-triazol-4-amine;
1H-triazole-1,5-diamine;
1H-triazole-1,3-diamine;
1H-1,2,4-triazol-3-amine;
1,5-bis(4chlorophenyl)-1H-1,2,4-triazol-3-amine;
1,5-bis(4-fluorophenyl)-1H-1,2,4-triazol-3-amine;
1,5-bis(4-methoxyphenyl)-1H-1,2,4-triazol-3-amine;
1,5-diethyl-1H-1,2,4-triazol-3-amine;
1,5-dimethyl-1H-1,2,4-triazol-3-amine;
1,5-diphenyl-1H-1,2,4-triazol-3-amine;
1-(4-chlorophenyl)-1H-1,2,4-triazol-3-amine;
1-ethyl-1H-1,2,4-triazol-3-amine;
1-methyl-1H-1,2,4-triazol-3-amine;
1-methyl-5-phenyl-1H-1,2,4-triazol-3-amine;
1-propyl-1H-1,2,4-triazol-3-amine;
5-(1-ethyl-1-methylpropyl)-1H-1,2,4-triazol-3-amine;
5-(1-methylethyl)-1H-1,2,4-triazol-3-amine;
5-(2-methylpropyl)-1H-1,2,4-triazol-3-amine;
5-(4-chlorophenyl)-1-(4-fluorophenyl)-1H-1,2,4-triazol-3-amine;
5-(4-chlorophenyl)-1-(4-methylphenyl)-1H-1,2,4-triazol-3-amine;
5-(4-methoxyphenyl)-1H-1,2,4-triazol-3-amine;
5-(3-methylphenyl)-1H-1,2,4-triazol-3-amine;
5-(4-methylphenyl)-1H-1,2,4-triazol-3-amine;
5-butyl-1H-1,2,4-triazol-3-amine;
5-ethyl-1H-1,2,4-triazol-3-amine;
5-ethyl-1-methyl-1H-1,2,4-triazol-3-amine;
5-hexyl-1H-1,2,4-triazol-3-amine;
5-methyl-1H-1,2,4-triazol-3-amine;
5-pentyl-1H-1,2,4-triazol-3-amine;
5-phenyl-1H-1,2,4-triazol-3-amine;
5-propyl-1H-1,2,4-triazol-3-amine; and
5-(4-chlorophenyl)-3-hydrazino-2-methyl-2H-1,2,4-triazole;

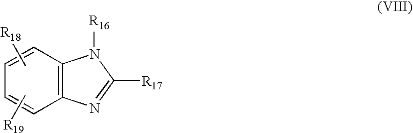

wherein R₁₆ is hydrogen or an amino group; R₁₇ is an amino group or a guanidino group when R₁₆ is hydrogen; or R₁₇ is an amino group when R₁₆ is an amino group; R₁₈ and R₁₉ are independently hydrogen, hydroxy, a lower alkyl group, a lower alkoxy group, or an aryl group; and their biologically or pharmaceutically acceptable acid or alkali addition salts.

The lower alkyl groups in the compounds of Formula VIII preferably contain 1-6 carbon atoms and include methyl, ethyl, propyl, butyl, pentyl, hexyl, and the corresponding branched chain isomers thereof. The lower alkoxy groups likewise contain 1-6, and preferably 1-3, carbon atoms, and are illustrated by methoxy, ethoxy, n-propoxy, isopropoxy and the like. The aryl groups encompassed by the above formula are those containing 6-10 carbon atoms, such as phenyl and lower alkyl substituted-phenyl, e.g., tolyl and xylyl, and phenyl substituted by 1-2 halo, hydroxy or lower alkoxy groups.

The halo atoms in the above Formula VIII may be fluoro, chloro, bromo or iodo.

The biologically or pharmaceutically acceptable salts of the compounds of Formula VIII are those tolerated by the mammalian body and include acid addition salts derived from a variety of organic and inorganic acids such as sulfuric, phosphoric, hydrochloric, sulfamic, citric, lactic, maleic, succinic, tartaric, cinnamic, acetic, benzoic, gluconic, ascorbic and related acids.

Of the compounds encompassed by Formula VIII, certain substituent are preferred. For instance, the compounds wherein R₁₇ is an amino group are a preferred group.

Representative of the compounds of Formula VIII are:
2-guanidinobenzimidazole;
1,2-diaminobenzimidazole;
1,2-diaminobenzimidazole hydrochloride;
2-aminobenzimidazole;
2-aminobenzimidazole-5-01;
4,5-dimethyl-2-aminobenzimidazole;
4,6-dichloro-2-aminobenzimidazole;
4,6-dimethyl-2-aminobenzimidazole;
4,7-dimethoxy-2-aminobenzimidazole;
4,7-dimethyl-2-aminobenzimidazole;
4-chloro-2-aminobenzimidazole;
5,6-dichloro-2-aminobenzimidazole;
5,6-diethoxy-2-aminobenzimidazole;
5,6diethoxy-2-aminobenzimidazole;
5,6-dimethyl-2-aminobenzimidazole;
5-butyl-2-aminobenzimidazole;
5-propyl-2-aminobenzimidazole;
5-ethyl-2-aminobenzimidazole;
5-methyl-2-aminobenzimidazole;
5-chloro-2-aminobenzimidazole;
5-fluoro-2-aminobenzimidazole;
5-methoxy-2-aminobenzimidazole;
5-chloro-6-methyl-2-aminobenzimidazole;
5-chloro-6-fluoro-2-aminobenzimidazole;
5-methyl-2-guanidinobenzimidazole;
4,6-methyl-2-guanidinobenzimidazole;
5,6-dimethyl-2-guanidinobenzimidazole;
5-chloro-2-guanidinobenzimidazole;
4,5-dichloro-2-guanidinobenzimidazole;
4,6-dichloro-2-guanidinobenzimidazole;
5-bromo-2-guanidinobenzimidazole;
5-methoxy-2-guanidinobenzimidazole;
5-methylbenzimidazole-1,2-diamine;
5-chlorobenzimidazole-1,2-diamine;
5,6-dimethylbenzimidazole-1,2-diamine; and
2,5-diaminobenzimidazole;
   R₂₀—CH—(NHR₂₁)—COOH (IX)

wherein R₂₀ is selected from the group consisting of hydrogen; lower alkyl, optionally substituted by one or two hydroxyl, thiol, phenyl, hydroxyphenyl, lower alkylthiol, carboxy, aminocarboxy or amino groups and R₂₁ is selected from the group of hydrogen and an acyl group; and their biocompatible and pharmaceutically acceptable acid addition salts.

The lower alkyl groups of the compounds of Formula IX contain 1-6 carbon atoms and include methyl, ethyl, propyl, butyl, pentyl, hexyl and the corresponding branched chain isomers thereof.

The acyl groups referred to herein are residues of lower alkyl, aryl and heteroaryl carboxylic acids containing 2-10 carbon atoms. They are typified by acetyl, propionyl, butanoyl, valeryl, hexanoyl and the corresponding higher chain and branched chain analogs thereof. The acyl radicals may also contain one or more double bonds and/or an additional acid functional group e.g., glutaryl or succinyl.

The amino acids utilized herein can possess either the L&D; stereochemical configuration or be utilized as mixtures thereof. However, the L-configuration is preferred.

Equivalent to the compounds of Formula IX for the purposes of this invention are the biocompatible and pharmaceutically acceptable salts thereof Such salts can be derived from a variety of inorganic and organic acids such as methanesulfonic, hydrochloric, toluenesulfonic, sulfuric, maleic, acetic, phosphoric and related acids.

Representative compounds of the compounds of Formula IX are: lysine; 2,3-diaminosuccinic acid; cysteine and the biocompatible and pharmaceutically acceptable salts thereof.

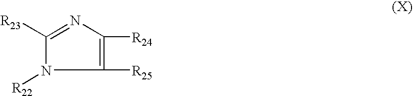

wherein R₂₂ and R₂₃ are independently hydrogen, an amino group or a mono- or di-amino lower alkyl group; R₂₄ and R₂₅ are independently hydrogen, a lower alkyl group, an aryl group; or an acyl group with the proviso one of R₂₂ and R₂₃ must be an amino group or an mono- or diamino lower alkyl group; and their biologically or pharmaceutically acceptable acid or alkali addition salts.

The lower alkyl groups of the compounds of Formula X contain 1-6 carbon atoms and include methyl, ethyl, propyl, butyl, pentyl, hexyl, and the corresponding branched-chain isomers thereof. The mono-or di-amino alkyl groups are lower alkyl groups substituted in the chain by one or two amino groups.

The aryl groups referred to herein encompass those containing 6-10 carbon atoms, such as phenyl and lower alkyl substituted-phenyl, e.g., tolyl and xylyl, and phenyl substituted by 1-2 halo, hydroxy and lower alkoxy groups. The acyl groups referred to herein are residues of lower alkyl, aryl and heteroaryl carboxylic acids containing 2-10 carbon atoms. They are typified by acetyl, propionyl, butanoyl, valeryl, hexanoyl and the corresponding higher chain and branched chain analogs thereof. The acyl radicals may also contain one or more double bonds and/or an additional acid functional group, e.g., glutaryl or succinyl.

The heteroaryl groups referred to above encompass aromatic heterocyclic groups containing 3-6 carbon atoms and one or more heteroatoms such as oxygen, nitrogen or sulfur.

The halo atoms in the above Formula X may be fluoro, chloro, bromo and iodo. The lower alkoxy groups contain 1-6, and preferably 1-3, carbon atoms and are illustrated by methoxy, ethoxy, propoxy, isopropoxy and the like. The term biologically or pharmaceutically acceptable salts refers to salts which are tolerated by the mammalian body and are exemplified by acid addition salts derived from a variety of organic and inorganic acids such as sulfuric, phosphoric, hydrochloric hydrobromic, hydroiodic, sulfamic, citric, lactic, maleic, succinic, tartaric, cinnamic, acetic, benzoic, glucomic, ascorbic and related acids.

Of the compounds encompassed by Formula X, certain combinations of substituents are preferred. For instance, when R₂₂ and R₂₃ are both amino groups, then R₂₄ and R₂₅ are preferably both hydrogen atoms. When R₂₂or R₂₃ is amino group and one of R₂₄ or R₂₅ is an aryl group, the other of R₂₄ and R₂₅ is preferably hydrogen.

Representative compounds of Formula X are:
1,2-diamino4-phenyl[1H]imidazole;
2-aminoimidazole sulfate;
1,2-diaminoimidazole;
1-(2,3-diaminopropyl)imidazole trihydrochloride;
4,5-diphenylimidazole-1,2-diamine;
4-(4-bromophenyl)imidazole-1,2-diamine;
4-(4-chlorophenyl)imidazole-1,2-diamine;
4-(4-hexylphenyl)imidazole-1,2-diamine;
4-(4-methoxyphenyl)imidazole-1,2-diamine,
4-phenyl-5-propylimidazole-1,2-diamine;
1-aminoimidazole;
1-amino4-methylimidazole;
1-amino4-(2-methylphenyl)imidazole;
2-aminoimidazole;
4-(2,4-dichlorophenyl)-2-aminoimidazole;
4-(4-methoxyphenyl)-2-aminoimidazole;
4-(4ethoxyphenyl)-2-aminoimidazole;
4-(4-fluorophenyl)-2-aminoimidazole;
4-(4-bromophenyl)-2-aminoimidazole;
4-butyl-2-aminoimidazole;
4-ethyl-2-aminoimidazole;
4-methyl-2-aminoimidazole;
4-phenyl-2-aminoimidazole;
4-propyl-2-aminoimidazole;
1-(3-aminopropyl)imidazole;
1-(3-amino-1-methylpropyl)imidazole;
1-(3-aminopropyl)4-5diethylimidazole;
1-(3-aminopropyl)4-methylimidazole;
1-(3-aminopropyl)4-phenylimidazole;
1-(3-aminopropyl)5-methyl4-phenylimidazole;
1-(2-aminoethyl)imidazole;
2-aminomethyl)imidazole;
2-(aminomethyl)-4-methylimidazole;
2-(3-aminopropyl)imidazole;
2-(3-aminopropyl)-4-5-dimethylimidazole;
2-(3-aminopropyl)-4-methylimidazole;
1,2-diamino-4-methylimidazole;
1,2-diamino4,5-dimethylimidazole; and
1,2-diamino-methyl-5-acetylimidazole;

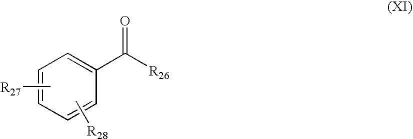

wherein R₂₆ is a hydroxy, lower alkoxy, amino, amino lower alkoxy, mono-lower alkylamino lower alkoxy, di-lower alkylamino lower alkoxy or hydrazino group, or a group of the formula —NR₂₉ R₃₀, wherein R₂₉ is hydrogen or lower alkyl, and R₃₀ is an alkyl group of 1-20 carbon atoms, an aryl group, a hydroxy lower alkyl group, a carboxy lower alkyl group, cycloloweralkyl group or a heterocyclic group containing 4-7 ring members and 1-3 heteroatoms; or R₂₉ and R₃₀ together with the nitrogen form a morpholino, piperidinyl, or piperazinyl group; or when R₂₉ is hydrogen, then R₃₀ can also be a hydroxy group;

R₂₇ is 0-3 amino or nitro groups, and/or a hydrazino group, a hydrazinosulfonyl group, a hydroxyethylamino-or an amidino group;

R₂₈ is hydrogen or one or two fluoro, hydroxy, lower alkoxy, carboxy, loweralkylamino, dilower alkylamino or a hydroxyloweralkylamino groups; with the proviso that when R₂₆ is hydroxy or lower alkoxy, then R₂₇ is a non-hydrogen substituent;

with the further proviso that when R₂₆ is hydrazino, then there must be at least two non-hydrogen substituents on the phenyl ring;

and with the further proviso that when R₂₈ is hydrogen, then R₃₀ can also be an aminoimino, guanidyl, aminoguanidinyl or diaminoguanidyl group;

their pharmaceutically acceptable salts and hydrates.

The lower alkyl groups of the compounds of Formula XI contain 1-6 carbon atoms and include methyl, ethyl, propyl, butyl, pentyl, hexyl, and the corresponding branched-chain isomers thereof. The cycloalkyl groups contain 4-7 carbon atoms and are exemplified by groups such as cyclobutyl, cyclopentyl, cyclohexyl, 4-methylcyclohexyl and cycloheptyl groups.

The heterocyclic groups of the compounds of Formula XI include 4-7 membered rings having at least one and up to 3 heteroatoms, e.g., oxygen, nitrogen, or sulfur, therein, and including various degrees of unsaturation.

Representatives of such heterocyclic groups are those such as morpholino, piperidino, homopiperidino, piperazino, methylpiperazino, hexamethylenimino, pyridyl, methylpyridyl, imidazolyl, pyrrolidinyl, 2,6-dimethylmorpholino, furfural, 1,2,4-triazoylyl, thiazolyl, thiazolinyl, methylthiazolyl, and the like.

Equivalent to the compounds of Formula XI for the purposes of this invention are the biocompatible and pharmaceutically acceptable salts and hydrates thereof. Such salts can be derived from a variety of organic and inorganic acids, including, but not limited to, methanesulfonic, hydrochloric, hydrobromic, hydroiodic, toluenesulfonic, sulfuric, maleic, acetic and phosphoric acids.

When the compounds of Formula XI contain one or more asymmetric carbon atoms, mixtures of enantiomers, as well as the pure (R) or (S) enantiomeric form can be utilized in the practice of this invention.

Of the compounds encompassed by Formula XI, certain combinations of substituents are preferred. For instance, when R₁ is a hydroxy, then R₂ is preferably one or two amino groups, or a single hydrazino or a single hydrazino-sulfonyl group. When R₁ is hydroxy and R₂ is a single amino group or a single hydrazino group, the R₂ substituent is preferably para to the carboxy substituent. When R is hydroxy and R₂ is two amino groups, they are preferably meta and para to the carboxy substituent. When R₁ is hydroxy and R₂ is a single hydrazino-sulfonyl group, then the R₂ substituent is preferably meta to the carboxy substituent.

When R₁ is a dialkyl aminolower alkoxy group, then R₂ is preferably a single amino group.

When R₁ is a carboxylower alkylamino group, then R₂ is preferably a single amino group.

In addition, compounds having a 3,4-diamino-, 3-hydroxy-4-amino-, 3-amino-4-hydroxy-, 3,5-diamino-4-hydroxy-, 3,5-diamino4-alkoxy- or 2,3-diamino-5-fluoro substituent pattern on the phenyl ring are highly preferred.

Representative compounds of the present invention are:
4-(cyclohexylamino-carbonyl)-o-phenylene diamine hydrochloride;
4-aminobenzhydrazide;
3,4-diaminobenzhydrazide;
4-(n-butylamino-carbonyl)-o-phenylene-diamine dihydrochloride;
4-(ethylamino-carbonyl)-o-phenylene-diamine dihydrochloride;
4-carbamoyl-o-phenylene diamine hydrochloride;
4-hydroxybenzhydrazide;
3-amino4-hydroxybenzoic acid;
4-amino-3-hydroxybenzoic acid;
3-amino-4-hydroxybenzhydrazide;
3-amino-4-hydroxybenzhydrazide dihydrochloride;
4-amidinobenzamide hydrochloride;
4-(morpholino-carbonyl)-o-phenylene-diamine hydrochloride;
4-[(4-morpholino)hydrazino-carbonyl]-o-phenylenediamine;
4-(1-piperidinylamino-carbonyl)-o-phenylenediamine dihydrochloride;
2,4-diamino-3-hydroxybenzoic acid;
3,5-diamino-4-hydroxybenzoic acid;
4-amino-3-hydroxybenzoic acid;
4-amino-3-hydroxybenzamide;
4,5-diamino-2-hydroxybenzoic acid;
3,4-diaminobenzamide;
3,4-diaminobenzhydrazide;
3,4-diamino-N,N-bis(1-methylethyl)benzamide;
3,4-diamino-N,N-diethylbenzamide;
3,4-diamino-N,N-dipropylbenzamide;
3,4-diamino-N-(2-furanylmethyl)benzamide
3,4-diamino-N-(2-methylpropyl)benzamide;
3,4-diamino-N-(4,5-dihydro-2-thiazolyl)benzamide;
3,4-diamino-N-(5-methyl-2-thiazolyl)benzamide;
3,4-diamino-N-(6-methoxy-2-benzothiazolyl)benzamide;
3,4-diamino-N-(6-methoxy-8-quinolinyl)benzamide;
3,4-diamino-N-(6-methyl-2-pyridinyl)benzamide;
3,4-diamino-N-(1H-benzimidazol-2-yl)benzamide;
3,4-diamino-N-(2-pyridinyl)benzamide;
3,4-diamino-N-(2-thiazolyl)benzamide;
3,4-diamino-N-(4-pyridinyl)benzamide;
3,4-diamino-N-[9H-pyrido(3,4-b)indol-6-yl]benzamide
3,4-diamino-N-butylbenzamide;
3,4-diamino-N-cyclohexylbenzamide;
3,4-diamino-N-cyclopentylbenzamide;
3,4-diamino-N-decylbenzamide;
3,4-diamino-N-dodecylbenzamide;
3,4-diamino-N-methylbenzamide;
3,4-diamino-N-octylbenzamide;
3,4-diamino-N-pentylbenzamide;
3,4-diamino-N-phenylbenzamide;
3-amino4-hydroxybenzamide;
3-amino4-hydroxy-N-octylbenzamide;
4-(diethylamino-carbonyl)-o-phenylene diamine;
4-(tert-butylamino-carbonyl)o-phenylene diamine;
4-isobutylamino-carbonyl)-o-phenylene diamine;
4-(neopentylamino-carbonyl)-o-phenylene diamine;
4-(dipropylamino-carbonyl)-o-phenylene diamine;
4-(n-hexylamino-carbonyl)-o-phenylene diamine;
4-(n-decylamino-carbonyl)-o-phenylene diamine;
4-(n-dodecylamino-carbonyl)-o-phenylene diamine;
4-(1-hexadecylamino-carbonyl)-o-phenylene diamine;
4-(octadecylamino-carbonyl)-o-phenylene diamine;
4-(hydroxylamino-carbonyl)-o-phenylene diamine;
4-(2-hydroxyethylamino-carbonyl)-o-phenylene diamine;
4-[(2-hydroxyethylamino)ethylamino-carbonyl]o-phenylene diamine;
4-[(2-hydroxyethyloxy)ethylamino-carbonyl]-o-phenylene diamine;
4-(6-hydroxyhexylamino-carbonyl)-o-phenylene diamine;
4-(3-ethoxypropylamino-carbonyl)-o-phenylene diamine;
4-(3-isopropoxypropylamino-carbonyl)-o-phenylene diamine;
4-(3-dimethylaminopropylamino-carbonyl)-o-phenylene diamine;
4-(N,N,2,2-tetramethyl-1,³-propanoamino-carbonyl)-o-phenylene diamine;
4-[4-(2-aminoethyl)morpholino-carbonyl]-o-phenylene diamine;
4-[4-(3-aminopropyl)morpholino-carbonyl]-o-phenylene diamine;
4-[N-(3-aminopropyl)pyrrolidino-carbonyl]-o-phenylene diamine;
4-[3-(N-piperidino)propylamino-carbonyl]-o-phenylene diamine;
4-[3-(4-methylpiperazinyl)propylamino-carbonyl]-o-phenylene diamine;
4-(3-imidazoylpropylamino-carbonyl)-o-phenylene diamine;
4-(3-phenylpropylamino-carbonyl)-o-phenylene diamine;
4-[2-(N,N-diethylamino)ethylamino-carbonyl]-o-phenylene diamine;
4-(imidazolylamino-carbonyl)-o-phenylene diamine;
4-(pyrrolidinyl-carbonyl)-o-phenylene diamine;
4-(piperidino-carbonyl)-o-phenylene diamine;
4-(1-methylpiperazinyl-carbonyl)-o-phenylene diamine;
4-(2,6-dimethylmorpholino-carbonyl)o-phenylene diamine;
4-(pyrrolidin-1-ylamino-carbonyl)-o-phenylene diamine;
4-(homopiperidin-1-ylamino-carbonyl)-o-phenylene diamine;
4-(4-methylpiperazine-1-ylamino-carbonyl)-o-phenylene diamine;
4-(1,2,4-triazol-1-ylamino-carbonyl)-o-phenylene diamine;
4-(guanidinyl-carbonyl)-o-phenylene diamine;
4-(guanidinylamino-carbonyl)-o-phenylene diamine;
4-(aminoguanidinylamino-carbonyl)-o-phenylene diamine;
4-(diaminoguanidinylamino-carbonyl)-o-phenylene diamine;
2-hydroxy4-(diethylamino-carbonyl)aniline;
2-hydroxy-4-(tertbutylamino-carbonyl)aniline;
2-hydroxy4-(isobutylamino-carbonyl)aniline;
2-hydroxy-4-(neopentylamino-carbonyl)aniline;
2-hydroxy4-(dipropylamino-carbonyl)aniline;
2-hydroxy4-(n-hexylamino-carbonyl)aniline;
2-hydroxy-4-(n-decylamino-carbonyl)aniline;
2-hydroxy-4-(n-dodecylamino-carbonyl)aniline;
2-hydroxy-4-(1-hexadecylamino-carbonyl)aniline;
2-hydroxy4-(octadecylamino-carbonyl)aniline;
2-hydroxy-4-(hydroxylamino-carbonyl)aniline;
2-hydroxy-4-(2-hydroxyethylamino-carbonyl)aniline;
2-hydroxy-4-((2-hydroxyethylamino)ethylamino-carbonyl) aniline;
2-hydroxy-4-((2-hydroxyethyloxy)ethylamino-carbonyl)aniline;
2-hydroxy-4-(6-hydroxyhexylamino-carbonyl)aniline;
2-hydroxy4-(3-ethoxypropylamino-carbonyl)aniline;
2-hydroxy4-(3-isopropoxypropylamino-carbonyl)aniline;
2-hydroxy-4-(3-dimethylaminopropyl amino-carbonyl)aniline;
2-hydroxy4-(N,N,2,2-tetramethyl-1,3-propanoamino-carbonyl)aniline;
2-hydroxy-4-(4-(2-aminoethyl)morpholino-carbonyl)aniline;
2-hydroxy-4-[4-(3-aminopropyl)morpholino-carbonyl]aniline;
2-hydroxy-4-[N-(3-aminopropyl)pyrrolidino-carbonyl]aniline;
2-hydroxy-4-[3-(N-piperidino)propylamino-carbonyl]aniline propane;
2-hydroxy-4-[3-(4-methylpiperazinyl)propylamino-carbonyl]aniline;
2-hydroxy-4-(3-imidazoylpropylamino-carbonyl)aniline;
2-hydroxy-4-(3-phenylpropylamino-carbonyl)aniline;
2-hydroxy-4-[2-(N,N-diethylamino)ethylamino-carbonyl]aniline;
2-hydroxy-4-(imidazolylamino-carbonyl)aniline;
2-hydroxy-4-(pyrrolidinyl-carbonyl)aniline;
2-hydroxy-4-(piperidino-carbonyl)aniline;
2-hydroxy-4-(1-methylpiperazinyl-carbonyl)aniline;
2-hydroxy-4-(2,6-dimethylmorpholino-carbonyl)aniline;
2-hydroxy4-(pyrrolidin-1-ylamino-carbonyl)aniline;
2-hydroxy4-(homopiperidin-1-ylamino-carbonyl)aniline;
2-hydroxy4-(4-methylpiperazine-1-ylamino-carbonyl)aniline;
2-hydroxy4-(1,2,4-triazol-1-ylamino-carbonyl)aniline;
2-hydroxy4-(guanidinyl-carbonyl)aniline;
2-hydroxy4-(guanidinylamino-carbonyl)aniline;
2-hydroxy4-(aminoguanidinylamino-carbonyl)aniline;
2-hydroxy4-(diaminoguanidinylamino-carbonyl)aniline;
3,5-diamino-hydroxybenzoic acid;
3,4,5-triaminobenzamide;
3,5-diaminosalicylic acid;
3,5-diamino-2-methylbenzoic acid;
5-aminoisophthalic acid;
3-aminophthalic acid;
3-hydroxyanthranilic acid;
3,5-diamino4-methyl benzoic acid;
3,4-aminosalicylic acid;
4-aminosalicylic acid;
5-aminosalicylic acid;
4-guanidinobenzoic acid;
4,5-difluoroanthranilic acid;
4-fluoro-3-nitrobenzoic acid;
3-amino-2,5,6-trifluorobenzoic acid;
2-fluoro-5-nitrobenzoic acid;.
methyl 3,5-diamino-4-hydroxy benzoate;
3-amino-5-nitrosalicylic acid;
2,4-dihydroxy-3,5-dinitrobenzoic acid;
3,5-dinitro4-hydroxybenzoic acid;
methyl 3,5-dinitro-4-hydroxybenzoate;
ethyl 3,5-dinitro-4-hydroxybenzoate;
3,5-dinitro-p-toluic acid;
3,5-dinitrosalicylic acid;
methyl 3-amino-5-nitrosalicylate;
2-amino-3-methylbenzoic acid;
4-amino-3-methoxybenzoic acid;
2-amino4,5-dimethoxybenzoic acid;
3,5-diaminobenzoic acid;
2-aminoterephthalic acid;
methyl 3,5diamino-2,4-dihydroxybenzoate;
3-amino4-fluorobenzoic acid;
isopropyl-3,5-dinitro-4-hydroxybenzoate;
methyl 4-aminosalicylate;
4-aminosalicylhydrazide;
4-(3,5-diamino-4-hydroxybenzoyl)-morpholine;
3,5-diamino-4-fluorobenzoic acid;
ethyl 3,5-diamino4-hydroxybenzoate;
isopropyl 3,5-diamino-2,4-dihydroxybenzoate;
5-amino-2-fluorobenzoic acid;
3,5-diamino-4-hydroxybenzanilide;
isopropyl 3,5-diamino4-methylbenzoate;
ethyl 3,5-diamino-4-ethoxybenzoate;
isopropyl 3,5-diamino4-isopropyloxybenzoate;
3,5-diamino4-methoxybenzoic acid;
3,5-diamino-4-methylbenzhydrazide;
3,5-diamino4-methoxybenzhydrazide;
isopropyl 3,4-diaminobenzoate;
methyl 3-amino-4-hydrazinobenzoate
5-aminosalicylic acid;
methyl 3,5-diamino-4-methoxybenzoate;
3,4-diaminobenzohydroxamic acid;
3,5-diamino-4-methylaminobenzoic acid;
3,5-diamino-4-isopropylaminobenzoic acid;
3,5-diamino-4-dimethylaminobenzoic acid;
methyl 3-amino4-fluorobenzoate;
3,5-diamino-4-isopropyloxybenzhydrazide;
isopropyl 3-amino4-hydrazinobenzoate;
3-amino4-fluorobenzhydrazide;
3,5-diamino-4-isopropyloxybenzoic acid;
3,5-diamino-hydroxyethylaminobenzoic acid;
3,4,5-triaminobenzoic acid;
2,3-diamino-5-fluoro-benzoic acid;
4-hydrazinobenzoic acid;
4-aminobenzoic acid 2-(diethylamino)ethyl ester monohydrochloride;
4-aminobenzoic acid;
N-(4-aminobenzoyl)glycine;
hydrazinosulfonylenzoic acid; and
3,4-diaminobenzoic acid; and
their pharmaceutically acceptable salts and hydrates;

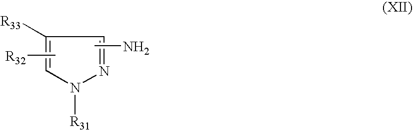

wherein R₃₁ is hydrogen, a lower alkyl or hydroxy group;

R₃₂ is hydrogen, hydroxy lower alkyl, a lower alkoxy group, a lower alkyl group, or an aryl group;

R₃₃ is hydrogen or an amino group;

and their biologically or pharmaceutically acceptable acid addition salts.

The lower alkyl groups of the compounds of Formula XII contain 5 1-6 carbon atoms and include methyl, ethyl, propyl, butyl, pentyl, hexyl, and the corresponding branched-chain isomers thereof. Likewise, the lower alkoxy groups contain 1-6, and preferably 1-3, carbon atoms and include methoxy, ethoxy, isopropoxy, propoxy and the like. The hydroxy lower alkyl groups include primary, secondary and tertiary alcohol substituent patterns.

The aryl groups of the compounds of Formula XII encompass those containing 6-10 carbon atoms, such as phenyl and lower alkyl substituted-phenyl, e.g., tolyl and xylyl, and phenyl substituted by 1-2 halo, hydroxy and lower alkoxy groups.

The halo atoms in the above Formula XII may be fluoro, chloro, bromo and iodo.

The term biologically or pharmaceutically acceptable salts refers to salts which are tolerated by the mammalian body and are exemplified by acid addition salts derived from a variety of organic and inorganic acids such as sulfuric, phosphoric, hydrochloric hydrobromic, hydroiodic, sulfuric, citric, lactic, maleic, succinic, tartaric, cinnamic, acetic, benzoic, gluconic, ascorbic and related acids.

Of the compounds encompassed by Formula XII, certain substituents are preferred. For instance, the compounds wherein R₃₂ is hydroxy and R₃₃ is an amino group are preferred.

Representative of the compounds of Formula XII are:
3,4-diaminopyrazole;
3,4-diamino-5-hydroxypyrazole;
3,4-diamino-5-methylpyrazole
3,4-diamino-5-methoxypyrazole;
3,4-diamino-5-phenylpyrazole;
1-methyl-3-hydroxy4,5-diaminopyrazole;
1-(2-hydroxyethyl)-3-hydroxy-4,5-diaminopyrazole;
1-(2-hydroxyethyl)-3-phenyl4,5-diaminopyrazole;
1-(2-hydroxyethyl)-3-methyl4,5-diaminopyrazole;
1-(2-hydroxyethyl)4,5-diaminopyrazole;
1-(2-hydroxypropyl)-3-hydroxy4,5-diaminopyrazole;
3-amino-5-hydroxypyrazole; and
1-2-hydroxy-2-methylpropyl)-3-hydroxy4,5-diaminopyrazole;
and their biologically and pharmaceutically acceptable acid addition salts;

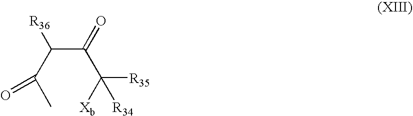

wherein X_{b} is oxygen or nitrogen; R₃₄ is hydrogen, lower alkyl or aryl;

R₃₅ is hydrogen, lower alkyl, lower alkenyl, aryl, or hydroxy lower alkyl;

R₃₆ is hydrogen, hydroxy, lower alkyl, aryl, halo, lower alkanoyl, or aryl lower alkyl; and their biologically or pharmaceutically acceptable salts with organic or inorganic bases.

The lower alkyl groups of the compounds of Formula XIII contain 1-6 carbon atoms and include methyl, ethyl, propyl, butyl, pentyl, hexyl, and the corresponding branched-chain isomers thereof. Similarly, the lower alkenyl groups referred to contain 2-6 carbon atoms and include ethenyl, propenyl and the like. The lower alkanoyl groups likewise contain 2-6 carbon atoms and are exemplified by acetyl, propionyl and the like.

The aryl groups of the compounds of Formula XIII encompass those containing 6-10 carbon atoms, such as phenyl and lower alkyl substituted-phenyl, e.g., tolyl and xylyl, and phenyl substituted by 1-2 halo, hydroxy and lower alkoxy groups.

The halo atoms in the above in Formula XIII may be fluoro, chloro, bromo and iodo. The lower alkoxy groups contain 1-6, and preferably 1-3, carbon atoms and are illustrated by methoxy, ethoxy, propoxy, isopropoxy and the like.

The term biologically or pharmaceutically acceptable salts refers to salts which are tolerated by the mammalian body and are exemplified by salts derived from a variety of organic and inorganic bases such as amines, e.g., procaine, or N,N′-dibenzylethylenediamine, or alkali or alkaline-earth metal salts, e.g., potassium or sodium hydroxide, calcium hydroxide, and the like. Thus formed are the amine salts or the corresponding alkali or alkaline-earth metal salts.

Of the compounds encompassed by Formula XIII, certain patterns of substituents are preferred. For instance, when R₃₆ is lower alkanoyl, then R₃₄ and R₃₅ are preferably hydrogen, lower alkyl or phenyl. Also preferred are the compounds wherein R₃₄, R₃₅ and R₃₆ are all hydrogen, or those wherein R₃₆ is hydrogen.

Representative of the compounds of Formula XIII are:
2,4-pyrrolidinedione;
2,4(3H,5H)-furandione (tetronic acid);
5-ethyl-2,4(3H,5H)-furandione;
5-methyl-2,4(3H,5H)-furandione;
5-propyl-2,4(3H,5H)-furandione;
5,5-dimethyl-2,4(3H,5H)-furandione;
5-hexyl-5-methyl-2,4(3H,5H)-furandione;
5-ethylidene-2,4(3H,5H)-furandione;
3-butyl-2,4(3H,5H)-furandione;
3-benzyl-2,4(3H,5H)-furandione;
3-ethyl-2,4(3H,5H)-furandione;
3-hydroxy-2,4(3H,5H)-furandione;
3-isobutyl-2,4(3H,5H)-furandione;
3-isopentyl-2,4(3H,5H)-furandione;
3-methyl-2,4(3H,5H)-furandione;
3-ethyl-5-methyl-2,4(3H,5H)-furandione;
3,5-di;methyl-2,4(3H,5H)-furandione;
3,5-diphenyl-2,4(3H,5H)-furandione;
3-methyl-5-(2-methyl-2-propenyl)-2,4(3H,5H)-furandione;
3-propyl-2,4(3H,5H)-furandione;
3-tert-butyl-2,4(3H,5H)-furandione;
3-fluoro-5-(4-methoxyphenyl)-2,4(3H,5H)-furandione;
3-hydroxy-5-methyl-2,4(3H,5H)-furandione;
3-hydroxy-5-phenyl-2,4(3H,5H)-furandione;
3-acetyl-2,4(3H,5H)-furandione;
3-acetyl-5,5-dimethyl-2,4(3H,5H)-furandione;
3-acetyl-5-ethyl-2,4(3H,5H)-furandione;
3-acetyl-5-methyl-2,4(3H,5H)-furandione;
(5R)-3-acetyl-5-methyl-2,4(3H,5H)-furandione;
(5S)-3-acetyl-5-methyl-2,4(3H,5H)-furandione;
3-acetyl-5-phenyl-2,4(3H,5H)-furandione; (tetramic acid)
5-(1-hydroxyethyl)-2,4-pyrrolidinedione;
5-(1-methylethyl)-2,4-pyrrolidinedione;
(S)-5-(1-methylethyl)-2,4-pyrrolidinedione;
5-(1-methylpropyl)-2,4-pyrrolidinedione;
(.+−.)4-(2-methylpropyl)-2,4-pyrrolidinedione;
5-(2-methylpentyl)-2,4-pyrrolidinedione;
(.+−.)-5-(2-methylpropyl)-2,4-pyrrolidinedione;
(.+−.)-5-(2-phenylethyl)-2,4-pyrrolidinedione;
(.+−.)-5-(3-methylbutyl)-2,4-pyrrolidinedione;
5-ethyl-2,4-pyrrolidinedione;
5-hexyl-2,4-pyrrolidinedione;
5-methyl-2,4-pyrrolidinedione;
5-phenyl-2,4-pyrrolidinedione;
3-ethyl-5-phenyl-1-p-tolyl-2,4-pyrrolidinedione;
3-acetyl-1,5-dimethyl-2,4-pyrrolidinedione;
3,5-diethyl-5-phenyl-2,4-pyrrolidinedione;
3,5-dimethyl-5-p-tolyl-2,4-pyrrolidinedione;
3,5-dimethyl-5-phenyl-2,4-pyrrolidinedione; and
3-ethyl-5-methyl-5-phenyl-2,4-pyrrolidinedione;
   H₂N—NR₄₀—C(═NR₃₉)—NR₃₇R₃₈ (XIV)

wherein R₃₇ is a lower alkyl group, or a group of the formula-NR₄₁R₄₂ wherein R₄₁ is hydrogen, and R₄₂ is a lower alkyl group or a hydroxy(lower)alkyl group; or R₄₁ and R₄₂ together with the nitrogen atom are a heterocyclic group containing 4-6 carbon atoms and, in addition to the nitrogen atom, 0-1 oxygen, nitrogen or sulfur atoms;

R₃₈ is hydrogen or an amino group;

R₃₉ is hydrogen or an amino group;

R₄₀ is hydrogen or a lower alkyl group;

with the proviso that at least one of R₃₈, R₃₉, and R₄₀ is other than hydrogen; and with the further proviso that R₃₇ and R₃₈ cannot both be amino groups; their pharmaceutically acceptable acid addition salts.

The lower alkyl groups of the compounds of Formula XIV contain 1-6 carbon atoms and include methyl, ethyl, propyl, butyl, pentyl, hexyl, and the corresponding branched-chain isomers thereof.

The heterocyclic groups formed by the N—R₄₁ R₄₂ group are 4-7 membered rings having at 0-1 additional heteroatoms, e.g., oxygen, nitrogen, or sulfur, therein, and including various degrees of unsaturation. Representatives of such heterocyclic groups are those such as morpholino, piperidino, hexahydroazepino, piperazino, methylpiperazino, hexamethylenimino, pyridyl, methylpyridyl, imidazolyl, pyrrolidinyl, 2,6-dimethylmorpholino, 1,2,4-triazoylyl, thiazolyl, thiazolinyl, and the like.

Equivalent to the compounds of Formula XIV for the purposes of this invention are the biocompatible and pharmaceutically acceptable salts thereof. Such salts can be derived from a variety of organic and inorganic acids, including, but not limited to, methanesulfonic, hydrochloric, hydrobromic, hydroiodic, toluenesulfonic, sulfuric, maleic, acetic and phosphoric acids.

When the compounds of Formula XIV contain one or more asymmetric carbon atoms, mixtures of enantiomers, as well as the pure (R) or (S) enantiomeric form can be utilized in the practice of this invention.

Of the compounds encompassed by Formula XIV, certain combinations of substituents are preferred. For instance, compounds wherein R₃₇ is a heterocylic group, and particularly a morpholino or a hexahydroazepino group, are highly preferred.

Representative of the compounds of Formula XIV are:
2-(2-hydroxy-2-methylpropyl)hydrazinecarboximidic hydrazide;
N-(4-morpholino)hydrazinecarboximidamide;
1-methyl-N-(4-morpholino)hydrazinecarboximidamide;
1-methyl-N-(4-piperidino)hydrazinecarboximidamide;
1-(N-hexahydroazepino)hydrazinecarboximidamnide;
N,N-dimethylcarbonimidic dihydrazide;
1-methylcarbonimidic dihydrazide;
2-(2-hydroxy-2-methylpropyl)carbohydrazonic dihydrazide; and
N-ethylcarbonimidic dihydrazide;
   (R₄₃HN═)CR₄₄—W—CR₄₅(═NHR₄₃) (XV)

wherein R₄₃ is pyridyl, phenyl or a carboxylic acid substituted phenyl group of the formula

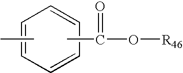

wherein R₄₆ is hydrogen, lower alkyl or a water-solubilizing ester moiety;

W is a carbon-carbon bond or an alkylene group of 1-3 carbon atoms, R₄₄ is a lower alkyl, aryl, or heteroaryl group and R₄₅ is hydrogen, a lower alkyl, aryl or heteroaryl group;

and their biologically or pharmaceutically acceptable acid addition salts.

The lower alkyl groups of the compounds of Formula XI preferably contain 1-6 carbon atoms and include methyl, ethyl, propyl, butyl, pentyl, hexyl, and the corresponding branched-chain isomers thereof. These groups are optionally substituted by one or more halo, hydroxy, amino or lower alkylamino groups.

The alkylene groups of the compounds of Formula XV likewise can be straight or branched chain, and are thus exemplified by ethylene, propylene, butylene, pentylene, hexylene, and their corresponding branched chain isomers.

In the R groups which are a carboxylic acid substituted phenyl group of the formula: 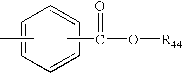
wherein R₄₄ is hydrogen, lower alkyl or a water-solubilizing ester moiety, the water solubilizing ester moiety can be selected from a variety of such esters known in the art. Typically, these esters are derived from dialkylene or trialkylene glycols or ethers thereof, dihydroxyalkyl groups, arylalkyl group, e.g., nitrophenylalkyl and pyridylalkyl groups, and carboxylic acid esters and phosphoric acid esters of hydroxy and carboxy-substituted alkyl groups. Particularly preferred water-solubilizing ester moieties are those derived from 2,3-dihydroxypropane, and 2-hydroxyethylphosphate.

The aryl groups encompassed by the above Formula XV are those containing 6-10 carbon atoms, such as phenyl and lower alkyl substituted-phenyl, e.g., tolyl and xylyl, and are optionally substituted by 1-2 halo, nitro, hydroxy or lower alkoxy groups.

Where the possibility exists for substitution of a phenyl or aryl ring, the position of the substituents may be ortho, meta, or para to the point of attachment of the phenyl or aryl ring to the nitrogen of the hydrazine group.

The halo atoms in the above Formula XV may be fluoro, chloro, bromo or iodo. The lower alkoxy groups contain 1-6, and preferably 1-3, carbon atoms and are illustrated by methoxy, ethoy, n-propoxy, isopropoxy and the like.

The heteroaryl groups in the above Formula XV contain 1-2 heteroatoms, i.e., nitrogen, oxygen or sulfur, and are exemplified by furyl, pyrrolinyl, pyridyl, pyrimidinyl, thienyl, quinolyl, and the corresponding alkyl substituted compounds.

For the purposes of this invention equivalent to the compounds of Formula XV are the biologically and pharmaceutically acceptable acid addition salts thereof. Such acid addition salts may be derived from a variety of organic and inorganic acids such as sulfuric, phosphoric, hydrochloric, hydrobromic, sulfamic, citric, lactic, maleic, succinic, tartaric, cinnamic, acetic, benzoic, gluconic, ascorbic, methanesulfonic and related acids.

Of the compounds encompassed by Formula XV, certain substituents are preferred. For instance, the compounds wherein W is a carbon-carbon bond, R₄₄ is a methyl group and R₄₅ is hydrogen are preferred.

Representative of the compounds of Formula XV are:
methyl glyoxal bis-(2-hydrazino-benzoic acid)hydrazone;
methyl glyoxal bis-(dimethyl 2-hydrazinobenzoate) hydrazone;
methyl glyoxal bis-(phenylhydrazine)hydrazone;
methyl glyoxal bis-(dimethyl 2-hydrazinobenzoate) hydrazone;
ethyl glyoxal bis-(4-hydrazinobenzoic acid)hydrazone;
methyl glyoxal bis-(dimethyl 4-hydrazinobenzoate)hydrazone;
methyl glyoxal bis-(2-pyridyl)hydrazone;
methyl glyoxal bis-(diethylene glycol methyl ether-2-hydrazinobenzoate) hydrazone;
methyl glyoxal bis-[1-(2,3-dihydroxypropane)-2-hydrazinebenzoate)hydrazone;
methyl glyoxal bis-[1-(2-hydroxyethane)-2-hydrazinobenzoate]hydrazone;
methyl glyoxal bis-[(1-hydroxymethyl-1-acetoxy))-2-hydrazino-2-benzoate]hydrazone;
methyl glyoxal bis-[(4-nitrophenyl)-2-hydrazinobenzoate]hydrazone;
methyl glyoxal bis-[(4-methylpyridyl)-2-hydrazinobenzoate]hydrazone;
methyl glyoxal bis-(triethylene glycol 2-hydrazinobenzoate)hydrazone; and
methyl glyoxal bis-(2-hydroxyethylphosphate-2-hydrazinebenzoate)hydrazone; 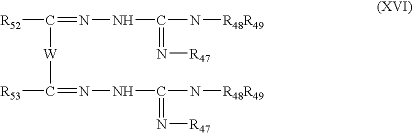
   wherein R₄₇ and R₄₈ are each hydrogen or, together, are an alkylene group of 2-3 carbon atoms, or, when R₄₇ is hydrogen, then R₄₉ can be a group of the formula
   alk-N—R₅₀R₅₁

wherein alk is a straight or branched chain alkylene group of 1-8 carbon atoms, and R₅₀ and R₅₁ are independently each a lower alkyl group of 1-6 carbon atoms, or together with the nitrogen atom form a morpholino, piperdinyl or methylpiperazinyl group;

R₄₉ is hydrogen, or when R₄₇ and R₄₈ are together an alkylene group of 2-3 carbon atoms, a hydroxyethyl group;

W is a carbon-carbon bond or an alkylene group of 1-3 carbon atoms, and R₅₂ is a lower alkyl, aryl, or heteroaryl group and R₅₃ is hydrogen, a lower alkyl, aryl or heteroaryl group; with the proviso that when W is a carbon-carbon bond, then R₅₂ and R₅₃ together can also be a 1,4-butylene group;

or W is a 1,2-, 1,3-, or 1,4-phenylene group, optionally substituted by one or two lower alkyl or amino groups, a 2,3-naphthylene group; a 2,5-thiophenylene group; or a 2,6-pyridylene group; and R₅₂ and R₅₃ are both hydrogen or a lower alkyl group;

or W is an ethylene group and R₅₂ and R₅₃ together are an ethylene group;

or W is an ethenylene group and R₅₂ and R₅₃ together are an ethenylene group;

or W is a methylene group and R₅₂ and R₅₃ together are a group of the formula ═C(—CH₃)—N—(H₃C—)C═ or —C—W—C— and R₅₂ and R₅₃ together form a bicyclo-(3,3,1)-nonane or a bicyclo-3,3,1-octane group and R₄₈ and R₄₈ are together an alkylene group of 2-3 carbon atoms and R₄₉ is hydrogen;

and their biologically or pharmaceutically acceptable acid addition salts.

The lower alkyl groups of the compounds of Formula XVI preferably contain 1-6 carbon atoms and include methyl, ethyl, propyl, butyl, pentyl, hexyl, and the corresponding branched-chain isomers thereof. These groups are optionally substituted by one or more halo hydroxy, amino or lower alkylamino groups.

The alkylene groups of the compounds of Formula XVI likewise can be straight or branched chain, and are thus exemplified by ethylene, propylene, butylene, pentylene, hexylene, and their corresponding branched chain isomers.

The aryl groups encompassed by the above Formula XVI are those containing 6-10 carbon atoms, such as phenyl and lower alkyl substituted-phenyl, e.g. tolyl and xylyl, and are optionally substituted by 1-2 halo, hydroxy or lower alkoxy groups.

The halo atoms in the above Formula XVI may be fluoro, chloro, bromo or iodo. The lower alkoxy groups contain 1-6, and preferably 1-3, carbon atoms and are illustrated by methoxy, ethoxy, n-propoxy, isopropoxy and the like.

The heteroaryl groups in the above Formula XVI contain 1-2 heteroatoms, i.e. nitrogen, oxygen or sulfur, and are exemplified by be furyl, pyrrolinyl, pyridyl, pyrimidinyl, thienyl, quinolyl, and the corresponding alkyl substituted compounds.

For the purposes of this invention equivalent to the compounds of Formula XVI are the biologically and pharmaceutically acceptable acid addition salts thereof. Such acid addition salts may be derived from a variety of organic and inorganic acids such as sulfuric, phosphoric, hydrochloric, hydrobromic, sulfamic, citric, lactic, maleic, succinic, tartaric, cinnamic, acetic, benzoic, gluconic, ascorbic, methanesulfonic and related acids.

Of the compounds encompassed by Formula XVI, certain substituents are preferred. For instance, the compounds wherein R₄₈ and R₄₉ are together an alkylene group of 2-3 carbon atoms are preferred. The compounds wherein R₅₂ and R₅₃ together are a butylene, ethylene, or an ethenylene group and those wherein R₅₂ and R₅₃ are both methyl or furyl groups are also highly preferred.

Representative of the compounds of Formula XVI are:
methyl glyoxal bis(guanylhydrazone);
methyl glyoxal bis(2-hydrazino-2-imidazoline-hydrazone);
terephthaldicarboxaldehyde bis(2-hydrazino-2-imidazoline hydrazone);
terephaldicarboxaldehyde bis(guanylhydrazone);
phenyl glyoxal bis(2-hydrazino-2-imidazoline hydrazone);
furyl glyoxal bis(2-hydrazino-2-imidazoline hydrazone);
methyl glyoxal bis>1-2-hydroxyethyl)-2-hydrazino-2-imidazoline hydrazone!;
methyl glyoxal bis>1-(2-hydroxyethyl)-2-hydrazino-1,4,5,6-tetrahydropyrimidine hydrazone!;
phenyl glyoxal bis(guanylhydrazone);
phenyl glyoxal bis) 1-(2-hydroxyethyl)-2-hydrazino-2-imidazoline hydrazone!;
furyl glyoxal bis>1-(2-hydroxyethyl)-2-hydrazino-2-imidazoline hydrazone!;
phenyl glyoxal bis>1-(2-hydroxyethyl)-2-hydrazino-1,4,5,6-tetrahydropyrimidine hydrazone!;
furyl glyoxal bis>1-(2-hydroxyethyl)-2-hydrazino-1,4,5,6-tetrahydropyrimidine hydrazone!;
2,3-butanedione bis(2-hydrazino-2-imidazoline hydrazone);
1,4-cyclohexane dione bis(2-hydrazino-2-imidazoline hydrazone!;
o-phthalic dicarboxaldehyde bis(2-hydrazino-2-imidazoline hydrazone);
2,3-naphthalene dialdehyde bis(2-hydrazino-2-imidazoline hydrazone);
1,2-cyclohexanedione bis(2-hydrazino-2-imidazoline hydrazone);
2,4-pentanedione bis(2-hydrazino-2-imidazoline hydrazone);
furyl bis(2-hydrazino-2-imidazoline hydrazone);
1,4-benzoquinone bis(2-hydrazino-2-imidazoline hydrazone);
methyl glyoxal bis(2-hydrazino-1,4,5,6-tetrahydropyrimidine hydrazone);
methyl glyoxal bis>N-(2,2-dimethyl-3-dimethylaminopropyl)hydrazine carboximidamide hydrazone!;
methyl glyoxal bis>N-3-dimethylaminopropyl)hydrazinecarboximidamnide hydrazone!;
methyl glyoxal bis{N-)2-(4-morpholino)ethyl!hydrazinecarboximidamide hydrazone}; and
methyl glyoxal bis{N->3-(4-methylpiperazin-1-yl)propyl!hydrazine carboximidamide hydrazone};
furyl glyoxal bis (guanyl hydrazone)dihydrochloride dihydrate;
2,3-pentanedione bis(2-tetrahydropyrimidine)hydrazone dihydrobromide;
1,2-cyclohexanedione bis(2-tetrahydropyrimidine)hydrazone dihydrobromide;
2,3-hexanedione bis(2-tetrahydropyrimidine)hydrazone dihydrobromide;
1,3-diacetyl bis(2-tetrahydropyrimidine)hydrazone dihydrobromide;
2,3-butanedione bis(2-tetrahydropyrimidine)hydrazone dihydrobromide;
2,6-diacetylpyridine-bis-(2-hydrazino-2-imidazoline hydrazone)dihydrobromide;
2,6-diacetylpyridine-bis-(guanyl hydrazone)dihydrochloride;
2,6-pyridine dicarboxaldehyde-bis-(2-hydrazino-2-imidazoline hydrazone)dihydrobromide trihydrate);
2,6-pyridine dicarboxaldehyde-bis(guanyl hydrazone)dihydrochloride;
1,4-diacetyl benzene-bis-(2-hydrazino-2-imidazoline hydrazone)dihydrobromide dihydrate;
1,3-diacetyl benzene-bis-(2-hydrazino-2-imidazoline)hydrazone dihydrobromide;
1,3-diacetyl benzene-bis(guanyl)-hydrazone dihydrochloride;
isophthalaldehyde-bis-(2-hydrazino-2-imidazoline)hydrazone dihydrobromide;
isophthalaldehyde-bis-(guanyl)hydrazone dihydrochloride;
2,6-diacetylaniline bis-(guanyl)hydrazone dihydrochloride;
2,6-diacetyl aniline bis-(2-hydrazino-2-imidazoline)hydrazone dihydrobromide;
2,5-diacetylthiophene bis(guanyl)hydrazone dihydrochloride;
2,5-diacetylthiophene bis-(2-hydrazino-2-imidazoline)hydrazone dihydrobromide;
1,4-cyclohexanedione bis(2-tetrahydropyrimidine)hydrazone dihydrobromide;
3,4-hexanedione bis(2-tetrahydropyrimidine)hydrazone dihydrobromide;
methyl glyoxal-bis-(4-amino-3-hydrazino-1,2,4-triazole)hydrazone dihydrochloride;
methyl glyoxal-bis-(4-amino-3-hydrazino-5-methyl-1,2,4-triazole)hydrazone dihydrochloride;
2,3 pentanedione-bis-(2-hydrazino-3-imidazoline)hydrazone dihydrobromide;
2,3-hexanedione-bis-(2-hydrazino-2-imidazoline)hydrazone dihydrobromide;
3-ethyl-2,4-pentane dione-bis-(2-hydrazino-2-imidazoline)hydrazone dihydrobromide;
methyl glyoxal-bis-(4-amino-3-hydrazino-5-ethyl-1,2,4-triazole)hydrazone dihydrochloride;
methyl glyoxal-bis-(4-amino-3-hydrazino-5-isopropyl-1,2,4-triazole)hydrazone dihy drochloride;
methyl glyoxal-bis-(4-amino-3-hydrazino-5-cyclopropyl-1,2,4-triazole)hydrazone dihydrochlori methyl glyoxal-bis-(4-amino-3-hydrazino-5-cyclobutyl-1,2,4-triazole)hydrazone dihydrochlorid 1,3-cyclohexanedione-bis-(2-hydrazino-2-imidazoline)hydrazone dihydrobromide;
3,5-diacetyl-1,4-dihydro-2,6-dimethyl pyridine bis(guanyl)hydrazone dihydrochloride;
3,5-diacetyl-1,4-dihydro-2,6-dimethyl pyridine bis-(2-hydrazino-2-imidazoline)hydrazone dihydrobrecmide;
bicyclo-(3,3,1 )nonane-3,7-dione bis-(2-hydrazino-2-imidazoline)hydrazone dihydrobromide; and
cis-bicyclo(3,3,1)octane-3,7-dione bis-(2-hydrazino-2-imidazoline) hydrazone dihydrobromide;

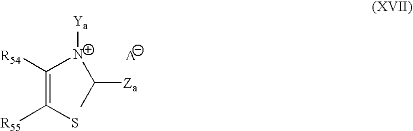

wherein R₅₄ and R₅₅ are independently selected from the group consisting of hydrogen, hydroxy(lower) alkyl, lower acyloxy(lower)alkyl, lower alkyl, or R₅₄ and R₅₃ together with their ring carbons may be an aromatic fused ring;

Zₐ is hydrogen or an amino group;

Yₐ is hydrogen, or a group of the formula —CH₂C(═O)—R₅₆ wherein R is a lower alkyl, alkoxy, hydroxy, amino or aryl group; or a group of the formula —CHR′

wherein R′ is hydrogen, or a lower alkyl, lower alkynyl, or aryl group; and

A is a halide, tosylate, methanesulfonate or mesitylenesulfonate ion.

The lower alkyl groups of the compounds of Formula XVII contain 1-6 carbon atoms and include methyl, ethyl, propyl, butyl, pentyl, hexyl, and the corresponding branched-chain isomers thereof. The lower alkynyl groups contain from 2 to 6 carbon atoms. Similarly, the lower alkoxy groups contain from 1 to 6 carbon atoms, and include methoxy, ethoxy, propoxy, butoxy, pentoxy, and hexoxy, and the corresponding branched-chain isomers thereof. These groups are optionally substituted by one or more halo, hydroxy, amino or lower alkylamino groups.

The lower acyloxy(lower)alkyl groups encompassed by the above Formula XVII include those wherein the acyloxy portion contain from 2 to 6 carbon atoms and the lower alkyl portion contains from 1 to 6 carbon atoms. Typical acyloxy portions are those such as acetoxy or ethanoyloxy, propanoyloxy, butanoyloxy, pentanoyloxy, hexanoyloxy, and the corresponding branched chain isomers thereof. Typical lower alkyl portions are as described hereinabove. The aryl groups encompassed by the above formula are those containing 6-10 carbon atoms, such as phenyl and lower alkyl substituted-phenyl, e.g., tolyl and xylyl, and are optionally substituted by 1-2 halo, hydroxy, lower alkoxy or di(lower)alkylamino groups. Preferred aryl groups are phenyl, methoxyphenyt and 4-bromophenyl groups.

The halo atoms in the above Formula XVII may be fluoro, chloro, bromo or iodo.

For the purposes of this invention, the compounds of Formula XVII are formed as biologically and pharmaceutically acceptable salts. Useful salt forms are the halides, particularly the bromide and chloride, tosylate, methanesulfonate, and mesitylenesulfonate salts. Other related salts can be formed using similarly non-toxic, and biologically and pharmaceutically acceptable anions.

Of the compounds encompassed by Formula XVII, certain substituents are preferred. For instance, the compounds wherein R₅₄ or R₅₅ are lower alkyl groups are preferred. Also highly preferred are the compounds wherein Yₐ is a 2-phenyl-2-oxoethyl or a 2-[4′-bromophenyl]-2-oxoethyl group.

Representative of the compounds of Formula XVII are:
3-aminothiazolium mesitylenesulfonate;
3-amino-4,5-dimethylaminothiazolium mesitylenesulfonate;
2,3-diaminothiazolinium mesitylenesulfonate;
3-(2-methoxy-2-oxoethyl)-thiazolium bromide;
3-(2-methoxy-2-oxoethyl)4,5-dimethylthiazolium bromide;
3-(2-methoxy-2-oxoethyl)-4-methylthiazolium bromide;
3-(2-phenyl-2-oxoethyl)-4-methylthizolium bromide;
3-(2-phenyl-2-oxoethyl)-4,5~diethylthiazolium bromide;
3-amino-4-methylthiazolium mesitylenesulfonate;
3(2-methoxy-2-oxoethyl)-5-methylthiazolium bromide;
3-(3-(2-phenyl-2-oxoethyl)-5-methylthiazolium bromide;
3-[2-(4′-bromophenyl)-2-oxoethyl]thiazolium bromide;
3-[2-(4′-bromophenyl)-2-oxoethyl]-4-methylthiazolium bromide;
3-[2-(4′-bromophenyl)-2-oxoethyl]-5-methylthiazolium bromide;
3-[2-(4′bromophenyl)-2-oxoethyl]-4,5-dimethylthiazolium bromide;
3-(2-methoxy-2-oxoethyl)-4methyl-5-(2-hydroxyethyl)thiazolium bromide;
3-(2-phenyl-2-oxoethyl)-4-methyl-5-(2-hydroxyethyl)thiazolium bromide;
3-[2-(4′-bromophenyl)-2-oxoethyl]4-methyl-5-(2-hydroxyethyl)thiazolium bromide;
3,4-dimethyl-5-(2-hydroxyethyl)thiazolium iodide;
3-ethyl-5-(2-hydroxyethyl)-4-methylthiazolium bromide;
3-benzyl-5-(2-hydroxyethyl)4-methylthiazolium chloride;
3-(2-methoxy-2-oxoethyl)benzothiazolium bromide;
3-(2-phenyl-2-oxoethyl)benzothiazolium bromide;
3-[2-(4′bromophenyl)-2-oxoethyl]benzothiazolium bromide;
3-(carboxymethyl)benzothiazolium bromide;
2,3-(diamino)benzothiazolium mesitylenesulfonate;
3-(2-amino-2-oxoethyl)thiazolium bromide;
3-(2-amino-2-oxoethyl)-4-methylthiazolium bromide;
3-(2-amino-2-oxoethyl)-5-methylthiazolium bromide;
3-(2-amino-2-oxoethyl)4,5-dimethylthiazolium bromide;
3-(2-amino-2-oxoethyl)benzothiazolium bromide;
3-(2-amino-2-oxoethyl) 4-methyl-5-(2-hydroxyethyl)thiazolium bromide;
3-amino-5-(2-hydroxyethyl)4-methylthiazolium mesitylenesulfonate;
3-(2-methyl-2-oxoethyl)thiazolium chloride;
3-amino-4-methyl-5-(2-acetoxyethyl)thiazolium mesitylenesulfonate;
3-(2-phenyl-2-oxoethyl)thiazolium bromide;
3-(2-methoxy-2-oxoethyl)4-methyl-5-(2-acetoxyethyl)thiazolium bromide;
3-(2-amino-2-oxoethyl)4-methyl-5-2-acetoxyethyl)thiazolium bromide;
2-amino-3-(2-methoxy-2-oxoethyl)thiazolium bromide;
2-amino-3-(2-methoxy-2-oxoethyl)benzothiazolium bromide;
2-amino-3-(2-amino-2-oxoethyl)thiazolium bromide;
2-amino-3-(2-amino-2-oxoethyl)benzothiazolium bromide;
3-[2-(4′-methoxyphenyl)-2-oxoethyl]-thiazolinium bromide;
3-[2-(2′,4′-dimethoxyphenyl)-2-oxoethyl]-thiazolinium bromide;
3-[2-(4′-fluorophenyl)-2-oxoethyl]-thiazolinium bromide;
3-[2-(2′,4′-difluorophenyl)-2-oxoethyl]-thiazolinium bromide;.
3-[2-(4′-diethylaminophenyl)-2-oxoethyl]-thiazolinium bromide;
3-propargyl-thiazolinium bromide;
3-propargyl4-methylthiazolinium bromide;
3-propargyl-5-methylthiazolinium bromide;
3-propargyl-4,5-dimethylthiazolinium bromide; and
3-propargyl4-methyl-5-(2-hydroxyethyl)-thiazolinium bromide;

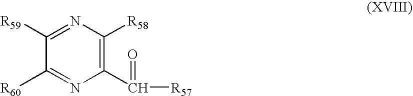

wherein

R₅₇ is OH, NHCONR₆₁R₆₂, or N═C(NR₆₁R₆₂₂)₂;

R₆₁ and R₆₂ are each independently selected from the group consisting of hydrogen; C₁₋₁₀ alkyl, straight or branched chain; aryl C₁₋₄ alkyl; and mono- or disubstituted aryl C₁₋₄ alkyl where the substituents are fluoro, chloro, bromo, iodo or C₁₋₁₀ alkyl, straight or branched chain;

R₅₈ and R₉ are each independently selected from the group consisting of hydrogen, amino, and mono- or di-substituted amino where the substituents are C₁₋₁₀ alkyl, straight or branched chain C₃₋₈, cycloalkyl; provided that R₁ and R₂ may not both be amino or substituted amino; and

R₆₀ is hydrogen, trifluoromethyl; fluoro; chloro; bromo; or iodo; or a pharmaceutically acceptable salt thereof.

The compounds to be used in the methods of this invention also include so-called nutraceuticals, for example, certain amino acids, vitamins or the like which are effective when administered individually, but preferably in combination, to bind to or otherwise interfere with the production of 3DG.

Representative examples of such substances are Vitamin C and lysine.

The methods of the present invention may also be practiced using antibodies or other immunoreactive substances that are capable of binding to 3DG precursors. These include, without limitation, the antibodies described in the above-mentioned U.S. Pat. Nos. 5,223,392, 5,494,791 and 5,518,720.

Pharmaceutical preparations which may be used in practicing the method of the present invention comprise one or more of the agents described above, as the active ingredient, in combination with a pharmaceutically acceptable carrier medium or auxiliary agent.

These ingredients may be prepared in various forms for administration, including both liquids and solids. Thus, the preparation may be in the form of tablets, caplets, pills or dragees, or can be filled in suitable containers, such as capsules, or, in the case of suspensions, filled into bottles. As used herein, “pharmaceutically acceptable carrier medium” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Representative examples of suitable carrier media include gelatine, lactose, starch, magnesium stearate, talc, vegetable and animal fats and oils, gum, polyalkylene glycol, or the like. Remington's Pharmaceutical Sciences, Fifteenth Edition, E. W. Martin (Mack Publishing Co., Easton, Pa. 1975) discloses various carriers used in formulating pharmaceutical compositions and known techniques for the preparation thereof. Except insofar as any conventional carrier medium is incompatible with the enzyme inhibitors of the invention, such as by producing any undesirable biological effect or otherwise interacting in a deleterious manner with any other component(s) of the pharmaceutical preparation, its use is contemplated to be within the scope of this invention.

In the pharmaceutical preparations of the invention, the active agent(s) may be present in an amount of at least 0.01% and generally not more than 10% by weight, based on the total weight of the preparation, including carrier medium and/or auxiliary agent(s), if any. Preferably, the proportion of active agent varies between 1.0% -5.0% by weight of the composition.

Anti-hypertensive drugs, including particularly the angiotensin-converting enzyme (ACE) inhibitors, may also be included as supplementary active agents in the pharmaceutical preparations of this invention.

Auxiliary agents, such as compounds that will protect the active agent from acid destruction in the stomach or facilitate the absorption of the active compound into the bloodstream can also be incorporated into the pharmaceutical preparation, if necessary or desirable. Such auxiliary agents may include, for example, complexing agents such as borate or other salts which partially offset the acid conditions in the stomach, and the like. Absorption can be increased by delivering the active compound as the salt of a fatty acid (in those cases where the active compound contains one or more basic functional groups).

The compounds of the invention, along with any supplementary active ingredient(s) may be administered, using any amount and any route of administration effective for binding to glycated protein, in vivo. Thus, the expression “therapeutically effective amount”, as used herein, refers to an amount of the therapeutic agent which is at once non-toxic and sufficient to provide the desired reduction in susceptibility to carcinoma associated with the intake of glycated protein. The exact amount required may vary, depending on the species, age, and general condition of the patient, the particular therapeutic agent and its mode of administration, and the like.

The agents used in the method of the invention are preferably formulated in dosage form for ease of administration and uniformity of dosage. Dosage unit form as used herein refers to a physically discrete unit of the therapeutic agent appropriate for the patient to be treated. Each dosage should contain the quantity of active material calculated to produce the desired therapeutic effect either as such, or in association with the selected pharmaceutical carrier medium. Typically, the small molecule agents used in practicing this invention will be administered in dosage units containing from about 25 mg to about 2500 mg of the compound, per dose, with a range of about 250 mg to about 750 mg being preferred.

The agents of the invention may be administered orally, parenterally, such as by intramuscular injection, intraperitoneal injection, intravenous infusion or the like, depending on the stability of the selected compounds to the various physiological conditions encounted in each route of administration. The small molecule agents may be administered orally or parenterally at dosage levels of about 1 mg to about 50 mg and preferably from about 10 mg to about 25 mg/kg, of patient body weight per day, one or more times a day, to obtain the desired therapeutic effect. Orally active agents are particularly preferred, provided the oral dose is capable of generating blood and/or target tissue levels of the above-described agents that are therapeutically active.

The normal concentration of immunoreactive agent used in the methods of this invention, including antibodies and antibody fragments, will be from about 1.0 mg and about 10 mg. These will typically be administered by intravenous or intraarterial infusion.

The compounds of the invention will typically be administered once per day or up to four times per day, depending upon the specific agent chosen. The exact regimen for administration of the compounds described herein will necessarily be dependent on the needs of the individual subject undergoing treatment, the type of therapy administered and the judgment of the attending physician. As used herein, the term “subject” includes both humans and animals.

While it is known that 3DG is an endogenously produced reactive dicarbonyl correlated with numerous pathologic conditions, it is not widely known that 3DG is present in various foodstuffs, the ingestion of which contributes to endogenous levels of 3DG. Examples of a number of commercial 3DG-containing syrups and a soft drink, along with the 3DG content thereof is presented in the following table.

TABLE 1

| | | |
|---|---|---|
| High Fructose Corn Syrup #1 | 2.65 | μmols/g |
| High Fructose Corn Syrup #2 | 2.26 | μmols/g |
| Karo ® Pancake Syrup | 380 | nmols/g |
| Karo ® Light Syrup | 340 | nmols/g |
| Crystal Light Syrup (Acme) | 57 | nmols/g |
| Aunt Jemima ® Pancake Syrup | 360 | nmols/g |
| Pepsi Cola ® | 0.54 | mM |

The production of high fructose corn syrup involves treating a solution of glucose with xylose isomerase. 3DG is produced as a by-product of this process. Furthermore, while catalyzing the reversible interconversion of D-xylose and D-xylulose, xylose isomerase (XI) has been observed by 13C-NMR spectroscopy to produce D-lyxose and 3-deoxy-D-xylosone (3-deoxy-D-glycero-pentose-2-ulose).

High fructose corn syrup solutions having reduced 3DG content may be obtained by treatment with an adsorbent resin or other suitable separation medium capable of selectively binding 3DG.

From preliminary observations on the ratios of 3DG and 3DF in the urine of diabetics and non-diabetics, diabetics appear to have reduced ability to detoxify 3DG of either endogenous or exogenous origin as well as a higher flux through the pathways that generate 3DG. These two factors combine to produce higher urinary concentrations of 3DG in diabetics (Lal et al., Arch. Biochem. and Biophys., 342(1): 254-60 (1997). See also the above-mentioned, published international patent application, WO 98/33492.

Although diabetic patients have significantly more 3DG in serum than do non-diabetic patients (12.78±2.49 μM versus 1.94±0.17 μM) (Toshimitsu Niwa et al., Nephron, 69: 438 (1995)), this compound is also found in healthy individuals. The body has developed clearance mechanisms for this molecule. One of these reactions is catalyzed by aldehyde reductase which detoxifies 3DG by reducing it to 3-deoxyfructose (3DF) which is efficiently excreted in the urine (Takahashi et al., Biochem., 34: 1433 (1995)). Another detoxification reaction oxidizes 3DG to 3-deoxy-2-ketogluconic acid (DGA_by oxoaldehyde dehydrogenase (Fujii et al., Biochem. Biophys. Res. Comm., 210: 852 (1995)).

Results of studies to date show that the efficiency of at least one of these enzymes, aldehyde reductase, is adversely affected in diabetes. When isolated from normal rat liver, a fraction of this enzyme is partially glycated on lysines 67, 84 and 140 and has low catalytic efficiency when compared with the normal, unmodified enzyme (Takahashi et al., Biochem., 34: 1433 (1995)). Since diabetic patients have higher ratios of glycated proteins than normoglycemic individuals they are likely to have both higher levels of 3DG and a reduced ability to detoxify this reactive molecule by reduction to 3DF. The ingestion of exogenous 3DG found in foodstuffs therefore represents an additional risk factor for the development and progression of complications in diabetic patients. Even in a clinically healthy individual, sufficient amounts of 3DG may exceed the physiologic capacity for detoxification. Thus, consumption of exogenous 3DG in foodstuffs is a risk factor for healthy individuals for the development of 3DG-related diseases and conditions.

The following examples are provided to describe the invention in further detail. These examples are provided for illustrative purposes only, and should in no way be construed as limiting the invention. All temperatures given in the examples are in degrees centigrade unless otherwise indicated.

### EXAMPLE 1

### Carcinogenic Effects of Fructoselysine Pathway

To investigate the carcinogenic potential of metabolites formed in the fructoselysine pathway, experiments have been conducted on a strain of rats with a high susceptibility to kidney carcinomas. Four rats were put on a glycated protein diet and three rats on a control diet. After ten weeks on the diet, the animals were sacrificed and their kidneys examined. In all four animals on the diet, kidney carcinomas of size greater than 1 mm were found, whereas no lesions this large were found in the control animals. The probability of this happening by chance is less than 2%. The data show that the elevated 3DG levels caused by the excess fructoselysine coming from the glycated protein in the animals diet found in the kidney tubular cells (known to be the cell of origin of most kidney carcinomas) can interact with the cellular DNA leading to a variety of mutagenic and ultimately carcinogenic events. The foregoing data indicate that this process is important in the development of human cancers in the kidney and elsewhere.

### EXAMPLE 2

### Dietary Effects of Glycated Protein Diet on Renal Cell Carcinoma in Susceptible Rats

In additional experiments assessing the relationship between a glycated protein diet and renal cell carcinoma, twenty-eight rats with a mutation making them susceptible to the development of kidney carcinoma were divided into two cohorts. One cohort was fed a glycated protein diet: the other cohort was on a control diet. The glycated protein diet consisted of a standard nutritious diet to which 3% glycated protein had been added. The glycated protein was made by mixing together casein and glucose (2:1) adding water (2× the weight of the dried material) and baking the mixture at 60° C. for 72 hours. The control was prepared in the same way except that no water was used and the casein and glucose were not mixed prior to baking. Rats were placed on the diets immediately following weaning at three weeks of age and maintained on the diets ad libitum for the next 16 weeks. The animals were then sacrificed, the kidneys fixed and nemotoxylin and eosin sections were made. These were examined for lesions by a trained pathologist. Four types of lesions were identified. These included: cysts, very small collections of tumor-like cells, typically less than 10 cells; small tumors, 0.5 mm or less, and tumors greater than 0.5 mm. For every type, more lesions were observed in the animals on the glycated diet than on the control diet as shown in the following table.

| | CYSTS | ≦10 CELLS | ≦0.5 mm | >.0.5 mm | TOTAL |
|---|---|---|---|---|---|
| CONTROL | 2 | 9 | 9 | 3 | 23 |
| GLYCATED | 9 | 21 | 32 | 6 | 68 |

To summarize the results, the average number of lesions per kidney section was computed for each diet. These were 0.82±0.74 and 2.43±2.33 in the control and-glycated diet, respectively. The likelihood of this happening by chance is about 2 in 100,000.

These results provide strong support for the premise that effects of the lysine recovery pathway, the discovery of which underlie the present invention, extend to causing mutations, and thus are an etiologic factor in the development of carcinoma, as well. These results provide a basis for the development of therapeutic methods and agents to counteract this pathway in order to reduce cancer in the kidney as well as in other organs where this pathway produces similar effects.

While certain embodiments of the present invention have been described and/or exemplified above, various other embodiments will be apparent to those skilled in the art from the foregoing disclosure. The present invention is, therefore, not limited to the particular embodiments described and/or exemplified, but is capable of considerable variation and modification without departure from the scope of the appended claims.

1. A method of reducing a susceptibility to tumor formation in a subject, caused by the presence in said subject of a substance selected from the group of 3-deoxyglucosone (3DG) and at least one precursor of 3 DG, comprising administering to said subject an agent which is effective to bind said 3 DG or said 3 DG precursor.

2. A method according to claim 1, wherein the tumor is renal cell carcinoma.

3. A method according to claim 1, wherein said agent is a compound selected from the group consisting of aminoguanidine and those having Formulas I-XVIII, as set forth in the foregoing specification.

4. A method according to claim 3, wherein said agent binds 3 DG.

5-32. (canceled)

33. A method according to claim 1, wherein said agent comprises aminoguanidine.
